(19) United States
(12) Patent Application Publication
(10) Pub. No.: US 2010/0216818 A1
(43) Pub. Date: Aug. 26, 2010

(54) Inhibitors of Post-Amadori Advanced Glycation End Products

(76) Inventor: Raja Khalifah, Durham, NC (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/775,658

(22) Filed: May 7, 2010

Related U.S. Application Data
(63) Continuation of application No. 10/651,481, filed on Aug. 29, 2003.
(60) Provisional application No. 60/407,465, filed on Aug. 30, 2002.

(51) Int. Cl. A61K 31/4439; C07D 401/04; A61K 31/444; A61K 31/506; A61P 27/02; A61P 43/00; A61P 19/02; A61P 3/10; A61P 9/00; A61P 3/00; A61P 13/12; A61P 9/12; A61P 3/06; A61P 9/10

(52) U.S. Cl. 514/256; 546/257; 514/334; 546/274.1; 514/341; 544/333

(57) ABSTRACT

The present invention provides compounds, pharmaceutical compositions, and methods for treating or inhibiting development of AGE- and/or ALE-associated complications in subjects in need thereof.

### CROSS REFERENCE

This application is a Continuation to U.S. Utility patent application Ser. No. 10/651,481, filed Aug. 29, 2003, which claims priority to U.S. Provisional Patent Application Ser. No. 60/407,465 filed Aug. 30, 2002, incorporated by reference herein in its entirety.

### FIELD OF THE INVENTION

This application relates to the fields of chemistry, medicine, renal disease, vascular disease, hyperlipidemia, hyperglycemia, advanced glycation end-products, and advanced lipoxidation end-products.

### BACKGROUND OF THE INVENTION

Advanced glycation end-products (AGEs) are carbohydrate-derived chemical modifications and crosslinks that accumulate in long-lived tissue proteins during normal aging. The increased rate of accumulation of AGEs during hyperglycemia is implicated in the development of long-term complications of diabetes, including but not limited to retinopathy, nephropathy, neuropathy, atherosclerosis, and cardiovascular disease. In addition, AGE formation has been implicated in a number of other pathologies, such as normal aging processes, arthritis, connective tissue disease, amyloidoses, and neurodegenerative amyloid diseases, such as Alzheimer's.

Advanced lipoxidation end products (ALEs) are lipid-derived chemical modifications and crosslinks that also accumulate in long-lived tissue proteins during normal aging, and are associated with hyperlipidemia, vascular disease, and renal disease in both diabetic and non-diabetic animal models. It is now recognized that some compounds, such as N^{ε}-(carboxymethyl)lysine (CML) and N^{ε}-(carboxyethyl)lysine (CEL), may be derived from either carbohydrates or lipids, leading to their designation as AGE/ALEs. Other compounds, such as pentosidine, appear to be true AGEs, while other compounds, such as malondialdehyde-lysine (MDA-Lys) and hydroxynonenal-lysine (HNE-Lys), are acknowledged to be ALEs, derived exclusively from lipids.

The elucidation of the pathogenic mechanisms of AGE and ALE-associated complications associated with hyperglycemia and/or hyperlipidemia is critical for developing rational therapy for their treatment and prevention. However, there is no consensus at present on the relative importance of the different possible pathogenic mechanisms that potentially contribute to these diabetic complications.

The compound pyridoxamine has recently been shown to inhibit both AGE and ALE formation in vitro, and to be useful for treating and preventing AGE and ALE-associated complications in hyperglycemic, hyperlipidemic, and hyperglycemic-hyperlipidemic animal models. (See, for example, U.S. Pat. No. 5,985,857; WO 00/21516; WO 00/23063) Such complications include, but are not limited to, diabetic nephropathy, proteinuria, impaired glomerular clearance, retinopathy, neuropathy, atherosclerosis, diabetes-associated hyperlipidemia, oxidative modification of proteins, urinary stone disease, obesity-related complications, proliferation or smooth muscle cells in the aorta, coronary artery occlusion, and hypertension; and dialysis-related disorders including dialysis-related cardiac morbidity and mortality, dialysis-related amyloidosis, dialysis-related increases in permeability of the peritoneal membrane in a dialysis patient, renal failure progression in a dialysis patient, and inhibiting ultrafiltration failure and peritoneal membrane destruction in a dialysis patient.

However, there remains a need in the art for further options to treat or inhibit development of AGE- and ALE-associated complications in patients in need thereof, particularly patients with hyperglycemia and/or hyperlipidemia.

### SUMMARY OF THE INVENTION

The present invention provides compounds, pharmaceutical compositions, and methods for treating or inhibiting development of AGE- and/or ALE-associated complications in a subject in need thereof. Thus, the invention provides novel compounds, detailed below, and pharmaceutical compositions thereof. In a preferred embodiment, the methods comprise administering one or more of the compounds or pharmaceutical compositions of the invention to subjects suffering from hyperglycemia and/or hyperlipidemia. The invention further comprises methods of treating or inhibiting development of disorders including diabetic nephropathy, proteinuria, impaired glomerular clearance, retinopathy, neuropathy, atherosclerosis, diabetes-associated hyperlipidemia, oxidative modification of proteins, arthritis, connective tissue diseases, amyloidosis, urinary stone disease, obesity-related complications, proliferation of smooth muscle cells in the aorta, coronary artery occlusion, and hypertension; and dialysis-related disorders including dialysis-related cardiac morbidity and mortality, dialysis-related amyloidosis, dialysis-related increases in permeability of the peritoneal membrane in a dialysis patient, renal failure progression in a dialysis patient, and inhibiting ultrafiltration failure and peritoneal membrane destruction in a dialysis patient. Said methods comprise administering an effective amount of one or more compounds of the present invention, or a pharmaceutically acceptable salt thereof, to a subject in need of such treatment.

### BRIEF DESCRIPTION OF THE FIGURES

FIG. 1 presents a synthetic scheme for [2,4′]Bipyridinyl-3′-ol (BST4944).

FIG. 2 presents a synthetic scheme for 5-Hydroxymethyl-4-(1H-imidazol-2-yl)-2-methyl-pyridin-3-ol (BST4997).

FIG. 3 presents a synthetic scheme for 3-Hydroxy-pyridine-4-carbaldehyde intermediate.

FIG. 4 presents a method for protecting the 3-OH during synthesis of 3-Hydroxy-pyridine-4-carbaldehyde intermediate.

FIG. 5 presents a synthetic scheme for 4-(1H-Imidazol-2-yl)-pyridin-3-ol (BST4996) from 3-Hydroxy-pyridine-4-carbaldehyde.

FIG. 6 presents synthetic schemes to produce other derivatives according to the invention. PG, PG1, and G2P refer to suitable protecting groups; LG refers to a suitable leaving group. (A) Production of 2′-halogen, 2′-secondary alcohol, and 2′-keto derivatives; (B) Production of 2′-alkenyl derivatives; (C) Production of 2′-hydroxymethyl and 2′-alkoxyalkyl derivatives (D) Production of 2′-methylamine derivatives; (E) Production of 3′-alkoxyalkyl and 3′-alkoxyl-5′-keto derivatives; (F) Production of 5′-keto-2′ methylhalogen derivatives; (G) Production of 5′-alkyl derivatives.

FIG. 7 details one method for modifying the hydroxymethyl group of BST-4997 to produce derivatives thereof

FIG. 8 details two methods for acylating the nitrogen atom in the imidazole ring of BST-4997 to provide various derivatives thereof.

FIG. 9 details a method for alkylating the nitrogen atom in the imidazole ring of BST-4997 by amino acid alkyl halides to provide various derivatives thereof.

FIG. 10 details one method for making mono- and di-substituted pyrimidine derivatives.

FIG. 11 details a method for making tri-substituted pyrimidine derivatives.

FIG. 12 details two methods for making substituted imidazole derivatives.

FIG. 13 is a graphical representation of the effect of BST-4997 on restoring nerve conduction velocity in streptozotocin diabetic rats.

FIG. 14 is a graphical representation of the effect of BST-4997 on restoring endoneurial perfusion in streptozotocin diabetic rats.

FIG. 15 is a graphical representation of the effect of BST-4997 on improving pain related measures in streptozotocin diabetic rats.

### DETAILED DESCRIPTION OF THE INVENTION

In one aspect, the invention is directed to compounds of Formula I:

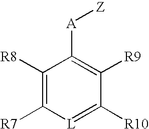

or pharmaceutically acceptable salts thereof, wherein
L is N, N⁺O⁻, or N⁺—Z with any counterion, wherein Z is C₁-C₆ alkyl;
A is a bond, C₁-C₄ alkyl, —O—C₁-C₄ alkyl, —C₁-C₄ alkyl-O—, C₁-C₄ alkoxy C₁-C₄ alkyl-, —N(R₂₀)C₁-C₄ alkyl, —C₁-C₄ alkyl-N(R₂₀)—, —C₁-C₂ alkyl-N(R₂₀)—C₁-C₂ alkyl, —S—C₁-C₄ alkyl, —C₁-C₄ alkyl-S—, or C₁-C₄ thioalkoxy C₁-C₄ alkyl-, wherein R₂₀ is H or C₁-C₄ alkyl;
R₈ is H, —CH₂OR₂ or OR₂;
R₉ is —CH₂OR₁ or OR₁;
R₁ and R₂ are independently H, C₁-C₆ alkyl, C₁-C₆ alkanoyl, C(O)NR₃R₄, C₁-C₆ alkoxy C₁-C₆ alkyl, arylalkyl or arylalkanoyl, wherein
the alkyl, alkanoyl and alkoxy groups are unsubstituted or substituted with 1, 2, or 3 groups that are independently hydroxy, C₁-C₄ alkoxy or NH₂; R₃ and R₄ are independently H, C₁-C₆ alkyl, C₁-C₆ alkoxy, arylalkyl, arylalkanoyl, or —CO₂alkyl, —CO₂alkylaryl; wherein
   the aryl portion of each arylalkyl or each arylalkanoyl is unsubstituted or substituted with 1, 2, 3, 4, 5 groups that are independently C₁-C₄ alkyl, C₁-C₄ alkoxy, hydroxy, halogen, haloalkyl, haloalkoxy, or nitro;
n is 0, 1, 2, or 3;
R₇ and R₁₀ are independently H, C₁-C₆ alkyl or C₂-C₈ alkenyl, each of which is unsubstituted or substituted by 1 or 2 groups that are independently hydroxy, halogen, NR₃R₄, alkoxy, heteroarylalkoxy, heterocycloalkylalkoxy, arylalkoxy, or aryl; wherein 1 or 2 carbons of the alkyl or alkenyl group can be replaced with a C(O) group or a CHO group;
   Z is heterocycloalkyl or heteroaryl, which is unsubstituted or substituted with 1, 2, 3, or 4 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, C₁-C₆ alkanoyl, hydroxy, hydroxy C₁-C₆ alkyl, NR₃R₄, or —C₁-C₆ alkyl NR₃R₄, wherein R₃ and R₄ are independently H, C₁-C₆ alkyl, C₁-C₆ alkoxy, arylalkyl, arylalkanoyl, or —CO₂alkyl, —CO₂alkylaryl;
   each alkyl, alkoxy, and alkanoyl group is unsubstituted or substituted with 1, 2, or 3, groups that are independently hydroxy or halogen,
   the aryl, heteroaryl, and heterocycloalkyl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro;
   any NH group in a heterocycloalkyl or heteroaryl group can optionally be NR₃, where R₃ is defined above;
provided that the Z group is attached to the CH₂ group or the pyridine ring through a carbon-carbon bond;
provided that when Z is tetrahydropyridine or piperidine, the Z group is attached via a carbon that is adjacent to a nitrogen atom.

As used herein, a “counterion” is a negatively charged ion, such as chloride, bromide, hydroxide, acetate, trifluoroacetate, perchlorate, nitrate, benzoate, maleate, sulfate, tartrate, hemitartrate, benzene sulfonate, and the like.

As used herein, the term “alkenyl” refers to a straight or branched hydrocarbon of a designed number of carbon atoms containing at least one carbon-carbon double bond. Examples of “alkenyl” include vinyl, allyl, and 2-methyl-3-heptene.

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

As used herein, the term “alkyl” includes straight or branched saturated hydrocarbons. C₁-C₆ alkyl refers to a straight or branched saturated hydrocarbon containing 1, 2, 3, 4, 5, or 6 carbon atoms. Examples of “alkyl” groups include methyl, ethyl, propyl, isopropyl, butyl, iso-, sec- and tert-butyl, pentyl, hexyl, heptyl, 3-ethylbutyl, and the like. Subgroups, such as, for example C₁-C₄ alkyl or C₃-C₅ are also contained within the above definition.

The term “alkanoyl” refers to a straight or branched alkyl group attached to the parent molecular moiety through a —C(O)— group. Examples of alkanoyl groups include, but are not limited to, acetyl and propionyl. A C₁-C₆ alkanoyl group is comprised of a C₁-C₆ alkyl group attached to the parent molecular moiety through a —C(O)— group. The term “arylalkanoyl” refers to an aryl group that is attached to the parent molecular moiety through an alkanoyl group. Examples of alkanoyl groups include, but are not limited to phenylacetyl, and phenylpropionyl. The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl. Preferred aryl groups have 6, 7, 8, 9, or 10 carbon atoms in the ring system.

The terms “halogen” or “halo” indicate fluorine, chlorine, bromine, and iodine.

The term “heterocycloalkyl,” refers to a non-aromatic ring system containing at least one heteroatom selected from nitrogen, oxygen, and sulfur. The heterocycloalkyl ring may be optionally fused to or otherwise attached to other heterocycloalkyl rings and/or non-aromatic hydrocarbon rings. Preferred heterocycloalkyl groups have from 3, 4, 5, 6, or 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “heterocycloalkylalkoxy” refers to a heterocycloalkyl group attached to the parent molecular moiety through an alkoxy group.

The term “heteroarylalkoxy” refers to a heteroaryl group attached to the parent molecular moiety through an alkoxy group.

Non-toxic pharmaceutically acceptable salts include, but are not limited to salts of inorganic acids such as hydrochloric, sulfuric, phosphoric, diphosphoric, hydrobromic, and nitric or salts of organic acids such as formic, citric, malic, maleic, fumaric, tartaric, succinic, acetic, lactic, methanesulfonic, p-toluenesulfonic, 2-hydroxyethylsulfonic, salicylic and stearic. Similarly, pharmaceutically acceptable cations include, but are not limited to sodium, potassium, calcium, aluminum, lithium and ammonium. Those skilled in the art will recognize a wide variety of non-toxic pharmaceutically acceptable addition salts.

The present invention also encompasses the acylated prodrugs of the compounds disclosed herein Those skilled in the art will recognize various synthetic methodologies, which may be employed to prepare non-toxic pharmaceutically acceptable addition salts and acylated prodrugs of the compounds of the present invention.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

The present invention also encompasses the prodrugs of the compounds disclosed herein. Those skilled in the art will recognize various synthetic methodologies that may be employed to prepare non-toxic pharmaceutically acceptable prodrugs of the compounds disclosed herein. Those skilled in the art will recognize a wide variety of non-toxic pharmaceutically acceptable solvates, such as water, ethanol, mineral oil, vegetable oil, and dimethylsulfoxide.

In a second embodiment of general formula I, the Z group contains at least one nitrogen atom.

In a third embodiment of general formula I,
A is C₁-C₄ alkyl, —O—C₁-C₄ alkyl, —C₁-C₄ alkyl-O—, C₁-C₄ alkoxy C₁-C₄ alkyl-, —N(R₂₀)C₁-C₄ alkyl, —C₁-C₄ alkyl-N(R₂₀)—, —C₁-C₂ alkyl-N(R₂₀)—C₁-C₂ alkyl, —S—C₁-C₄ alkyl, —C₁-C₄ alkyl-S—, or C₁-C₄ thioalkoxy C₁-C₄ alkyl-, wherein R₂₀ is H or C₁-C₄ alkyl;
R₇ and R₁₀ are independently H, C₁-C₆ alkyl or C₂-C₈ alkenyl, each of which is unsubstituted or substituted by 1 or 2 groups that are independently hydroxy, NR₃R₄, heteroarylalkoxy, heterocycloalkylalkoxy, arylalkoxy, or aryl; wherein 1 or 2 carbons of the alkyl or alkenyl group can be replaced with a C(O) group or a CHO group; and
Z is quinazoline; quinoxaline; imidazole; benzimidazole; piperazine; morpholine; thiomorpholine; quinoline; isoquinoline; 3-, 4-, 5-, 6-, 7-, or 8-tetrahydroisoquinoline; 1,2,4-triazole; hexahydropyridazine; tetrahydropyridazine; pyrazole; pyrrole; pyrimidine; pyrazine; isothiazole; 4(3H)-pyrimidinone; isoxazole; 1,3,5-triazine; hexahydropyrimidine; furan; tetrahydrofuran; tetrahydropyrimidine; piperidine; tetrahydropyridine; indole; indoline; benzoxazole; 1H-1,2,3-triazole; azocine; or imidazolidine; each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, C₁-C₆ alkanoyl, hydroxy, hydroxy C₁-C₆ alkyl, NR₃R₄, or —C₁-C₆ alkyl NR₃R₄, wherein R₃ and R₄ at each occurrence areas defined above and
   each alkyl, alkoxy, and alkanoyl group is unsubstituted or substituted with 1, 2, or 3, groups that are independently hydroxy or halogen,
   the aryl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro,
   any NH group in a heterocycloalkyl or heteroaryl group can optionally be NR₃, where R₃ is defined above;
provided that the Z group is attached to the CH₂ group through a carbon-carbon bond;
provided that when Z is tetrahydropyridine or piperidine, the Z group is attached via a carbon that is adjacent to a nitrogen atom.

In a fourth embodiment, which is a preferred version of the third embodiment,
A is C₁-C₄ alkyl, —O—C₁-C₄ alkyl, —C₁-C₄ alkyl-O—, C₁-C₄ alkoxy C₁-C₄ alkyl-, —N(R₂₀)C₁-C₄ alkyl, —C₁-C₄ alkyl-N(R₂₀)—, —C₁-C₂ alkyl-N(R₂₀)—C₁-C₂ alkyl, —S—C₁-C₄ alkyl, —C₁-C₄ alkyl-S—, or C₁-C₄ thioalkoxy C₁-C₄ alkyl-, wherein R₂₀ is H or C₁-C₄ alkyl; and Z contains at least two nitrogen atoms.

In a fifth embodiment, which is a preferred version of the fourth embodiment,
R₇ and R₁₀ are independently H, C₁-C₆ alkyl, which is unsubstituted or substituted by 1 or 2 groups that are independently hydroxy, NR₃R₄, heteroarylalkoxy, heterocycloalkylalkoxy, arylalkoxy, or aryl; and
Z is quinazoline; quinoxaline; imidazole; benzimidazole; piperazine; 1,2,4-triazole; hexahydropyridazine; tetrahydropyridazine; pyrazole; pyrimidine; pyrazine; 4(3H)-pyrimidinone; 1,3,5-triazine; hexahydropyrimidine; tetrahydropyrimidine; tetrahydropyridine; indole; indoline; 1H-1,2,3-triazole; or imidazolidine, each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, alkanoyl, hydroxy C₁-C₆ alkyl, NR₃R₄, or —C₁-C₆ alkyl-NR₃R₄, wherein
   R₃ and R₄ at each occurrence are independently H, C₁-C₆ alkyl, C₁-C₆ alkoxy, arylalkyl, arylalkanoyl, C₁-C₆ alkanoyl, —CO₂alkyl, —CO₂alkylaryl
   each alkyl, alkoxy, and alkanoyl group is unsubstituted or substituted with 1, 2, or 3, groups that are independently hydroxy or halogen,
   the aryl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro,
   any NH group in a heterocycloalkyl or heteroaryl group can optionally be NR₃, where R₃ is defined above;
provided that the Z group is attached to the CH₂ group or the pyridine ring through a carbon-carbon bond;
provided that when Z is tetrahydropyridine or piperidine, the Z group is attached via a carbon that is adjacent to a nitrogen atom.

In a sixth embodiment, which is a preferred version of the fifth embodiment
R₇ and R₁₀ are independently H, C₁-C₆ alkyl which is unsubstituted or substituted by 1 or 2 groups that are independently hydroxy, or NR₃R₄; wherein 1 or 2 carbons of the alkyl or alkenyl group can be replaced with a C(O) group or a CHO group; and
Z is imidazole; benzimidazole; piperazine; 1,2,4-triazole; hexahydropyridazine; tetrahydropyridazine; pyrazole; pyrimidine; pyrazine; hexahydropyrimidine; tetrahydropyrimidine; tetrahydropyridine; indole; indoline; 1H-1,2,3-triazole; or imidazolidine, each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, phenyl C₁-C₆ alkyl, phenyl C₁-C₆ alkanoyl, phenyl C₁-C₆ alkoxy, hydroxy C₁-C₆ alkyl, NR₃R₄, or —C₁-C₆ alkyl-NR₃R₄, wherein
   R₃ and R₄ are independently H, C₁-C₆ alkyl, benzyl, benzoyl, C₁-C₆ alkanoyl, —CO₂alkyl, —CO₂alkylphenyl;
   each alkyl, alkoxy, and alkanoyl group is unsubstituted or substituted with 1, 2, or 3, groups that are independently hydroxy, fluoro, or chloro;
   the phenyl groups are unsubstituted or substituted with 1, 2, 3, 4, or 5 groups that are C₁-C₄ alkyl, C₁-C₄ alkoxy, hydroxy, fluoro, chloro, CF₃, OCF₃, or nitro;
   any NH group in a heterocycloalkyl or heteroaryl group can optionally be NR₃, where R₃ is defined above;
provided that the Z group is attached to the CH₂ group through a carbon-carbon bond;
provided that when Z is tetrahydropyridine or piperidine, the Z group is attached via a carbon that is adjacent to a nitrogen atom.

In a seventh embodiment, which is a preferred version of the third embodiment, R₁ and R₂ are independently H, C₁-C₆ alkoxy C₁-C₆ alkyl, arylalkyl or arylalkanoyl, wherein
the aryl portion of each arylalkyl or each arylalkanoyl is unsubstituted or substituted with 1, 2, 3, 4, 5 groups that are independently C₁-C₄ alkyl, C₁-C₄ alkoxy, hydroxy, halogen, haloalkyl, haloalkoxy, or nitro; and
the alkyl, alkanoyl and alkoxy groups are independently substituted with 1, 2, or 3 groups that are independently hydroxy, C₁-C₄ alkoxy or NH₂.

In an eight embodiment, which is a preferred version of the sixth embodiment, R₁ and R₂ are independently hydrogen, C₁-C₄ alkyl, or benzyl, wherein
the phenyl portion of each benzyl is unsubstituted or substituted with 1, 2, or 3, groups that are independently C₁-C₄ alkyl, C₁-C₄ alkoxy, hydroxy, fluoro, chloro, CF₃, OCF₃, or nitro; and
the alkyl groups are independently substituted with 1, or 2, groups that are independently hydroxy, methoxy, ethoxy, propoxy, isopropoxy or NH₂.

In a ninth embodiment, which is a preferred version of the fourth embodiment,
R₇ and R₁₀ are independently H, C₁-C₆ alkyl, which is unsubstituted or substituted by 1 or 2 groups that are independently hydroxy, or NR₃R₄, heteroarylalkoxy, heterocycloalkylalkoxy, arylalkoxy, or aryl;
   the aryl, heteroaryl, or heterocycloalkyl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro,

A tenth embodiment, which is a preferred version of general formula I, is a compound of the formula:

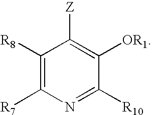

In an eleventh embodiment, which is a preferred version of the tenth embodiment, the Z group contains at least one nitrogen atom.

In a twelfth embodiment, which is a preferred version of the eleventh embodiment,
Z is quinazoline; quinoxaline; imidazole; benzimidazole; piperazine; morpholine; thiomorpholine; quinoline; isoquinoline; 3, 4, 5, 6, 7, or 8-tetrahydroisoquinoline; 1,2,4-triazole; hexahydropyridazine; tetrahydropyridazine; pyrazole; pyrimidine; pyrazine; isothiazole; 4(3H)-pyrimidinone; isoxazole; 1,3,5-triazine; hexahydropyrimidine; furan; tetrahydrofuran; tetrahydropyrimidine; piperidine; tetrahydropyridine; indole; indoline, benzoxazole; 1H-1,2,3-triazole; azocine; or imidazolidine; each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo C₁-C₆ alkyl, halo C₁-C₆ alkyl, aryl C₁-C₆ alkyl, aryl C₁-C₆ alkanoyl, aryl C₁-C₆ alkoxy, alkanoyl, hydroxy C₁-C₆ alkyl, NR₃R₄, or —C₁-C₆ alkyl NR₃R₄, wherein
   R₃ and R₄ are independently H, C₁-C₆ alkyl, C₁-C₆ alkoxy, arylalkyl, arylalkanoyl, C₁-C₆ alkanoyl, —CO₂alkyl, —CO₂alkylaryl;
   each alkyl, alkoxy, and alkanoyl group is unsubstituted or substituted with 1, 2, or 3, groups that are independently hydroxy or halogen,
   the aryl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro,
   any NH group in a heterocycloalkyl or heteroaryl group can optionally be NR₃, where R₃ is defined above;
provided that the Z group is attached to the pyridine ring through a carbon-carbon bond;
provided that when Z is tetrahydropyridine or piperidine, the Z group is attached via a carbon that is adjacent to a nitrogen atom;
provided that when Z is hexahydropyrimidine, it is substituted with two or three groups.

In a thirteenth embodiment, which is a preferred version of the twelfth embodiment, Z contains at least two nitrogen atoms.

In a fourteenth embodiment, which is a preferred version of the thirteenth embodiment
Z is quinazoline; quinoxaline; imidazole; benzimidazole; piperazine; 1,2,4-triazole; hexahydropyridazine; tetrahydropyridazine; pyrazole; pyrimidine; pyrazine; 4(3H)-pyrimidinone; 1,3,5-triazine; hexahydropyrimidine; tetrahydropyrimidine; tetrahydropyridine; indole; indoline; 1H-1,2,3-triazole; or imidazolidine, each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, alkanoyl, hydroxy C₁-C₆ alkyl, NR₃R₄, or —C₁-C₆ alkyl-NR₃R₄, wherein
   R₃ and R₄ are independently H, C₁-C₆ alkyl, C₁-C₆ alkoxy, arylalkyl, arylalkanoyl, C₁-C₆ alkanoyl, —CO₂alkyl, —CO₂alkylaryl
   each alkyl, alkoxy, and alkanoyl group is unsubstituted or substituted with 1, 2, or 3, groups that are independently hydroxy or halogen,
   the aryl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro,
   any NH group in a heterocycloalkyl or heteroaryl group can optionally be NR₃, where R₃ is defined above;
provided that the Z group is attached to the pyridine ring through a carbon-carbon bond;
provided that when Z is hexahydropyrimidine, it is substituted with two or three groups.

In a fifteenth embodiment, which is a preferred version of the fourteenth embodiment,
Z is imidazole; benzimidazole; piperazine; 1,2,4-triazole; hexahydropyridazine; tetrahydropyridazine; pyrazole; pyrimidine; pyrazine; hexahydropyrimidine; tetrahydropyrimidine; tetrahydropyridine; indole; indoline; 1H-1,2,3-triazole; or imidazolidine, each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, phenyl C₁-C₆ alkyl, phenyl C₁-C₆ alkanoyl, phenyl C₁-C₆ alkoxy, hydroxy C₁-C₆ alkyl, NR₃R₄, or —C₁-C₆ alkyl-NR₃R₄, wherein
   R₃ and R₄ are independently H, C₁-C₆ alkyl, benzyl, benzoyl, C₁-C₆ alkanoyl, —CO₂alkyl, —CO₂alkylphenyl;
   each alkyl, alkoxy, and alkanoyl group is unsubstituted or substituted with 1, 2, or 3, groups that are independently hydroxy, fluoro, or chloro;
   the phenyl groups are unsubstituted or substituted with 1, 2, 3, 4, or 5 groups that are C₁-C₄ alkyl, C₁-C₄ alkoxy, hydroxy, fluoro, chloro, CF₃, OCF₃, or nitro;
   any NH group in a heterocycloalkyl or heteroaryl group can optionally be NR₃, where R₃ is defined above;
provided that the Z group is attached to the pyridine ring through a carbon-carbon bond;
provided that when Z is hexahydropyrimidine, it is substituted with two or three groups.

In a sixteenth embodiment, which is a preferred version of the fifteenth embodiment,
R₁ and R₂ are independently H, C₁-C₆ alkoxy C₁-C₆ alkyl, arylalkyl or arylalkanoyl, wherein
   the aryl portion of each arylalkyl or each arylalkanoyl is unsubstituted or substituted with 1, 2, 3, 4, 5 groups that are independently C₁-C₄ alkyl, C₁-C₄ alkoxy, hydroxy, halogen, haloalkyl, haloalkoxy, or nitro; and
   the alkyl, alkanoyl and alkoxy groups are independently substituted with 1, 2, or 3 groups that are independently hydroxy, C₁-C₄ alkoxy or NH₂.

In a seventeenth embodiment, which is a preferred version of the sixteenth embodiment,

R₁ and R₂ are independently hydrogen, C₁-C₄ alkyl, or benzyl, wherein
the phenyl portion of each benzyl is unsubstituted or substituted with 1, 2, or 3, groups that are independently C₁-C₄ alkyl, C₁-C₄ alkoxy, hydroxy, fluoro, chloro, CF₃, OCF₃, or nitro; and
the alkyl groups are independently substituted with 1, or 2, groups that are independently hydroxy, methoxy, ethoxy, propoxy, isopropoxy or NH₂.

In an eighteenth embodiment, which is a further embodiment of the first embodiment, the compounds of the invention are directed to compounds of the formula:

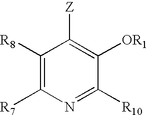

or pharmaceutically acceptable salts thereof, wherein
R₈ is H, —CH₂OR₂ or OR₂;
R₁ and R₂ are independently H, C₁-C₆ alkyl, C₁-C₆ alkanoyl, C₁-C₆ alkoxy C₁-C₆ alkyl, arylalkyl or arylalkanoyl, wherein
   the alkyl, alkanoyl and alkoxy groups are unsubstituted or substituted with 1, 2, or 3 groups that are independently hydroxy, C₁-C₄ alkoxy or NH₂;
R₃ and R₄ are independently H, C₁-C₆ alkyl, C₁-C₆ alkoxy, arylalkyl, arylalkanoyl, or —CO₂alkyl, —CO₂ alkylaryl; wherein
   the aryl portion of each arylalkyl or each arylalkanoyl is unsubstituted or substituted with 1, 2, 3, 4, or 5 groups that are independently C₁-C₄ alkyl, C₁-C₄ alkoxy, hydroxy, halogen, haloalkyl, haloalkoxy, or nitro;
R₇ and R₁₀ are independently H, C₁-C₆ alkyl or C₂-C₈ alkenyl, each of which is unsubstituted or substituted by 1 or 2 groups that are independently hydroxy, halogen, NR₃R₄, alkoxy, heteroarylalkoxy, heterocycloalkylalkoxy, arylalkoxy, or aryl; wherein 1 or 2 carbons of the alkyl or alkenyl group can be replaced with a C(O) group or a CHO group;
Z is heterocycloalkyl or heteroaryl containing 1, 2, or 3 nitrogen atoms, which is unsubstituted or substituted with 1, 2, 3, or 4 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, C₁-C₆ alkanoyl, hydroxy, hydroxy C₁-C₆ alkyl, NR₃R₄, or —C₁-C₆ alkyl NR₃R₄, wherein R₃ and R₄ are as defined above;
   each alkyl, alkoxy, and alkanoyl group is unsubstituted or substituted with 1, 2, or 3, groups that are independently hydroxy or halogen,
   the aryl, heteroaryl, and heterocycloalkyl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro;
      any NH group in a heterocycloalkyl or heteroaryl group can optionally be NR₃, where R₃ is defined above.

In a preferred embodiment of the eighteenth embodiment, Z is heteroaryl. In an even more preferred embodiment of the eighteenth embodiment, the heteroaryl is selected from the group consisting of pyridine, imidazole, diazole, and triazole, wherein said heteroaryl is unsubstituted or substituted as described for the eighteenth embodiment.

In a further preferred embodiment of the eighteenth embodiment, the Z group is attached to the pyridine ring through a carbon-carbon bond.

In a further preferred embodiment of the eighteenth embodiment, Z contains 2 nitrogen atoms and is substituted as described for the eighteenth embodiment. In an even more preferred embodiment, Z contains an unsubstituted nitrogen atom on both sides of the attachment point of Z.

In another preferred embodiment of the eighteenth embodiment, the aryl substituents on Z are phenyl or phenyl derivatives.

In a preferred embodiment of all of the various embodiments of the compounds of the invention, the L group is N. In a further preferred embodiment of all of the various embodiments of the compounds of the invention, R₉ is OR'.

Specific compounds according to these various embodiments include [2,4′]Bipyridinyl-3′-ol, 4-(1H-Imidazol-2-yl)-pyridin-3-ol, 5-Hydroxymethyl-4-(1H-imidazol-2-yl)-2-methyl-pyridin-3-ol, and other compounds as discussed below.

In a further aspect, the present invention provides pharmaceutical compositions comprising one or more compounds of the invention, as disclosed above and a pharmaceutically acceptable carrier. Preferred embodiments of the pharmaceutical compositions are described below.

In a further aspect, the present invention provides methods for treating or inhibiting development of one or more AGE- and/or ALE-associated complications in subject in need thereof comprising administering one or more compounds or pharmaceutical compositions of the invention to a subject in need thereof. As used herein, the phrase “AGE and/or ALE associated complications” includes, but is not limited to accelerated protein aging, retinopathy, nephropathy, proteinuria, impaired glomerular clearance, neuropathy, hyperlipidemia, hypertriglyceridemia, hypercholesterolemia, atherosclerosis, cardiovascular disease, and neurodegenerative amyloid diseases, such as Alzheimer's disease, diabetes-associated hyperlipidemia, oxidative modification of proteins, arthritis, connective tissue diseases, amyloidosis, urinary stone disease, obesity-related complications proliferation or smooth muscle cells in the aorta, coronary artery occlusion, and hypertension; and dialysis-related disorders including dialysis-related cardiac morbidity and mortality, dialysis-related amyloidosis, dialysis-related increases in permeability of the peritoneal membrane in a dialysis patient, renal failure progression in a dialysis patient, and inhibiting ultrafiltration failure and peritoneal membrane destruction in a dialysis patient.

In a further aspect, the invention provides methods for treating or inhibiting development of one or more of diabetic nephropathy, proteinuria, impaired glomerular clearance, retinopathy, neuropathy, atherosclerosis, diabetes-associated hyperlipidemia, oxidative modification of proteins, arthritis, connective tissue diseases, amyloidosis, urinary stone disease, obesity-related complications proliferation or smooth muscle cells in the aorta, coronary artery occlusion, and hypertension; and dialysis-related disorders including dialysis-related cardiac morbidity and mortality, dialysis-related amyloidosis, dialysis-related increases in permeability of the peritoneal membrane in a dialysis patient, renal failure progression in a dialysis patient, and inhibiting ultrafiltration failure and peritoneal membrane destruction in a dialysis patient, wherein the methods comprise administering an effective amount of one or more compounds of the present invention, or a pharmaceutically acceptable salt thereof, to a subject in need of such treatment. In a preferred embodiment, the methods are used to treat patients suffering from hyperlipidemia and/or hyperglycemia or their complications, or to inhibit development of complications arising from hyperlipidemia and/or hyperglycemia, such as those described above. While the methods of this aspect of the present invention are not limited by a specific mechanism, it is believed that the compounds of the invention are useful in treating or inhibiting development of these complications based on their ability to inhibit AGE and/or ALE formation, and thus to inhibit the development or progression of complications associated with accumulation of AGEs and/or ALEs.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, the term “inhibiting development of” means to prevent or to minimize development of the disorder or complication in individuals at risk of developing the disorder or complication.

The instant compounds can be administered individually or in combination, usually in the form of a pharmaceutical composition. Such compositions are prepared in a manner well known in the pharmaceutical art and comprise at least one active compound.

The compounds of the invention can be administered as the sole active pharmaceutical agent, or they can be used in combination with one or more other compounds useful for carrying out the methods of the invention, including but not limited to pyridoxamine, aminoguanidine, and agents that promote glycemic control, such as insulin, metformin, and thiazolidinediones. When administered as a combination, the therapeutic agents can be formulated as separate compositions that are given at the same time or different times, or the therapeutic agents can be given as a single composition.

The compounds may be made up in a solid form (including granules, powders or suppositories) or in a liquid form (e.g., solutions, suspensions, or emulsions). The compounds of the invention may be applied in a variety of solutions and may be subjected to conventional pharmaceutical operations such as sterilization and/or may contain conventional adjuvants, such as preservatives, stabilizers, wetting agents, emulsifiers, buffers etc.

The compounds of the invention may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of the invention and a pharmaceutically acceptable carrier. One or more compounds of the invention may be present in association with one or more non-toxic pharmaceutically acceptable carriers and/or diluents and/or adjuvants, and if desired other active ingredients. The pharmaceutical compositions containing compounds of the invention may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds and pharmaceutical compositions of the present invention may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds and pharmaceutical compositions of the present invention may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.01 mg to about 50 mg per kilogram of body weight per day, and more preferably between 0.1 mg to about 50 mg per kilogram of body weight per day, are useful in the treatment of the above-indicated conditions. The amount of active ingredient that may be combined with the carrier materials to produce a single dosage form will vary depending upon the host treated and the particular mode of administration. Dosage unit forms will generally contain between from about 1 mg to about 500 mg of an active ingredient.

Pharmaceutical compositions containing the compounds described herein are administered to an individual in need thereof. In a preferred embodiment, the subject is a mammal; in a more preferred embodiment, the subject is a human. In therapeutic applications, compositions are administered in an amount sufficient to carry out the methods of the invention. Amounts effective for these uses depend on factors including, but not limited to, the nature of the compound (specific activity, etc.), the route of administration, the stage and severity of the disorder, the weight and general state of health of the subject, and the judgment of the prescribing physician. The active compounds are effective over a wide dosage range. However, it will be understood that the amount of the compound actually administered will be determined by a physician, in the light of the above relevant circumstances. Therefore, the above dosage ranges are not intended to limit the scope of the invention in any way.

For administration to non-human mammals, the composition may also be added to the animal feed or drinking water. It may be convenient to formulate these animal feed and drinking water compositions so that the animal ingests an appropriate quantity of the composition during a meal or throughout the course of the day. It may also be convenient to present the composition as a premix for addition to the feed or drinking water.

The starting materials and various intermediates may be obtained from commercial sources, prepared from commercially available organic compounds, or prepared using well-known synthetic methods.

Representative examples of methods for preparing specific embodiments of the invention are set forth below.

### Example 1

### Synthesis of [2,4′]Bipyridinyl-3′-ol (BST4944)

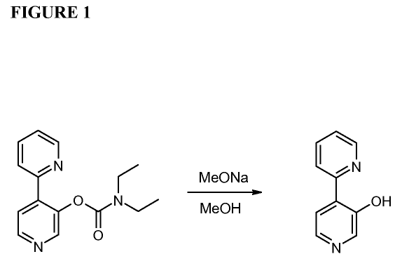

The synthetic scheme for [2,4′]Bipyridinyl-3′-ol is provided in FIG. 1. Diethyl-carbamic acid [2,4′]bipyridinyl-3′-yl ester (FW: 271, 395 mg, 1.45 mmol) was refluxed for two hours in MeOH (3 ml) to which sodium methoxide was added (800 μl). The solution was then kept for 2 hours at room temperature, followed by removal of solvent. After removal of solvent, the residue was re-dissolved in EtOAc and water and then neutralized to pH 7 with diluted H₂SO₄. Extractions with 10:1 EtOAc/MeOH were dried over MgSO₄, filtered and concentrated to afford 292 mg of product in oil.

### Physical Properties

### Compound C₁₀H₈N₂O, FW: 172.19

### Purification Method Extraction and Dryness

### Purity: 75%

TABLE 1
Spectral properties

| Compound | | pH2.0 | pH7.4 | pH9.4 |
|---|---|---|---|---|
| BST 4944 | λₘₐₓ₁ (nm) | 266 | 266 | 266 |
| | εₘₐₓ₁ (×10⁻³) | 1.54 | 1.52 | 1.38 |
| | λₘₐₓ₂ (nm) | 273 | 273 | 273 |
| | εₘₐₓ₂ (×10⁻³) | 1.71 | 1.68 | 1.43 |
| | λₘₐₓ₃ (nm) | 284 | 288 | 288 |
| | εₘₐₓ₃ (×10⁻³) | 1.84 | 1.46 | 1.14 |
| | λₘₐₓ₄ (nm) | 293 | 317 | 319 |
| | εₘₐₓ₄ (×10⁻³) | 1.75 | 1.19 | 1.09 |
| | λₘₐₓ₅ (nm) | 322 | | |
| | εₘₐₓ₅ (×10⁻³) | 1.34 | | |

### Example 2

### Synthesis of 5-Hydroxymethyl-4-(1H-imidazol-2-yl)-2-methyl-pyridin-3-ol (BST4997)

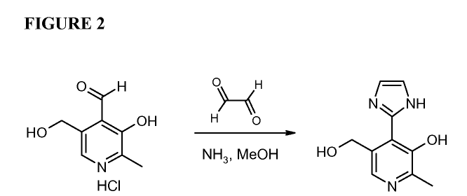

The synthetic scheme for 5-Hydroxymethyl-4-(1H-imidazol-2-yl)-2-methyl-pyridin-3-ol is provided in FIG. 2. Pyridoxal hydrochloride (251 mg, 1.24 mmol) was dissolved in MeOH (3 mL) followed by adding glyoxal (40% in water) (1 mL) and ammonium hydroxide (NH₄OH) (conc. 1 mL). The reaction solution was stirred for 16 hours. After removal of solids by filtration, the reaction solution was rotovaped to remove MeOH and purified by flash column with EtOAc/MeOH (8:1) as eluent (R_{f} 0.52), yield, 179 mg, 0.88 mmol, 71%.

### Physical Properties

### Compound: C₁₀H₁₁N₃O₂, FW: 205.22

### Purification Method: Flash Column

### Purity: 99%

TABLE 2
Spectral Properties

| Compound | | pH 2.0 | pH 7.4 | pH 9.4 |
|---|---|---|---|---|
| BST 4997 | λₘₐₓ₁ (nm) | 246 | 251 | 245 |
| | εₘₐₓ₁ (×10⁻³) | 7.01 | 7.56 | 7.57 |
| | λₘₐₓ₂ (nm) | 291-301 (flat) | 305 | 279-285 (flat) |
| | εₘₐₓ₂ (×10⁻³) | 3.98 | 3.83 | 3.64 |
| | λₘₐₓ₃ (nm) | 375 | 364 | 351 |
| | εₘₐₓ₃ (×10⁻³) | 3.41 | 5.53 | 4.95 |

### Example 3

### Synthesis of Intermediate Compound 3-Hydroxy-pyridine-4-carbaldehyde

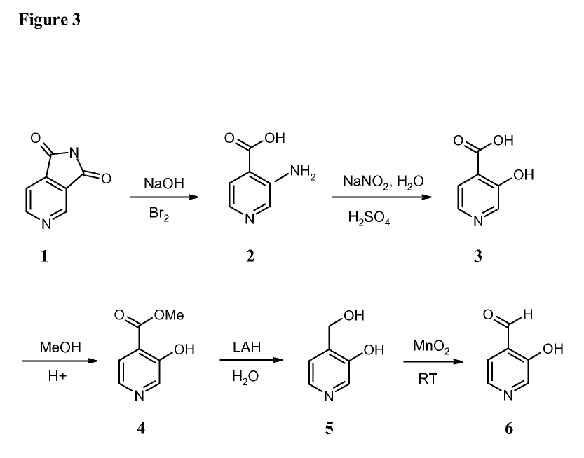

The synthetic scheme for 3-Hydroxy-pyridine-4-carbaldehyde is provided in FIG. 3. Hydration followed by Hofmann rearrangement of 3,4-pyridinedicarboximide (1) gave 3-amino-isonicotinic acid (2). Diazotization followed by hydrolysis of 3-amino-isonicotinic acid (2) afforded 3-hydroxy-isonicotinic acid (3). By Fischer esterification, 3-hydroxy-isonicotinic acid (3) was converted to 3-hydroxy-isonicotinic acid methyl ester (4), which was reduced to 4-hydroxymethyl-pyridin-3-ol (5) and then oxidized to 3-hydroxy-pyridine-4-carbaldehyde (6).

3-Amino-isonicotinic acid (2): Bromine (214 g, 1.34 mol) was slowly added into pre-cooled (5° C.) sodium hydroxide (10%, 3160 g) followed by adding 3,4-pyridinedicarboximide (195 g, 1.32 mol). The solution was heated to 80° C. and stirred for 1 hour. After cooling to 37° C., the solution was adjusted to pH 5.5 with addition of acetic acid (AcOH) (225 mL) and slowly stirred for 16 hours at 0° C. The solid was filtered off, washed with water, then with methanol (MeOH). The product 3-amino-isonicotinic acid (2) was dried to a light brown solid, yield, 112 g, 0.814 mol, 61%.

3-Hydroxy-isonicotinic acid (3): 3-Amino-isonicotinic acid (2) (112 g, 0.814 mol) was dissolved in deionized water (1800 mL) containing sulfuric acid (H₂SO₄) (90 mL) by warming to 52° C. and then cooled down to 8° C. (solids came back out). A solution of sodium nitrite (NaNO₂) (62.1 g) in deionized water (540 mL) was slowly added over 20 min while maintaining a temperature of 8-10° C. The slurry was heated to 82° C. and then cooled to 65° C. AcOH (90 mL) and ammonium hydroxide (NH₄OH) (˜150 mL) were added to adjust the pH to 4.5. The reaction mixture was further cooled to 0° C. and stirred for 16 hours. The product, 3-hydroxy-isonicotinic acid (3), was collected by filtration as a tan solid, yield, 105 g, 0.755 mol, 93%.

3-Hydroxy-isonicotinic acid methyl ester (4): 3-Hydroxy-isonicotinic acid (3) (104 g, 0.748 mol) was refluxed with MeOH (212 mL, 5.23 mol), H₂SO₄ (60 mL, 1.12 mol) and 1,2-dichloroethane (360 mL) for 20 hours. The reaction mixture was cooled to room temperature and diluted with deionized water. After removal of solid by filtration, the aqueous layer was basified with sodium bicarbonate (NaHCO₃) and refiltered. The organic layer was removed, and the aqueous layer was extracted with chloroform (CHCl₃) (×3). The combined organic layers were dried over magnesium sulfate (MgSO₄), filtered and concentrated to afford an off-white solid, yield, 90 g, 0.588 mol, 78%.

4-Hydroxymethyl-pyridin-3-ol (5): 3-Hydroxy-isonicotinic acid methyl ester (4) (1 g, 6.5 mmol) was dissolved in ether (20 mL, anhydrous). A suspension of lithium aluminum hydride (LAH) (248 mg) in ether (20 mL) was slowly added at 0° C. The mixture was stirred at room temperature for 6 hours until TLC showed reaction to be complete. Ethyl acetate (EtOAc) was added to quench the reaction and water was added to dissolve salts. After extraction with chloroform and ethyl acetate, the product stayed in the aqueous phase.

3-Hydroxy-pyridine-4-carbaldehyde (6): The mixture of 4-hydroxymethyl-pyridin-3-ol (5) (1 g, 6.19 mmol) and manganese oxide (MnO₂) (7 g) with TEA (861 μL) was stirred at room temperature in chloroform (CHCl₃) (50 mL, anhydrous) for 20 hours. The resulting material was filtered through a celite, washed with CHCl₃ and EtOAc, rotovaped to dryness, and re-dissolved in deionized water. Then the solution was extracted with CHCl₃ (×2), EtOAc (×2), and EtOAc/MeOH (10:1, ×5). The combined organics were dried over MgSO₄ and followed by rotovap. 220 mg of 3-hydroxy-pyridine-4-carbaldehyde (6) was obtained (˜30% yield).

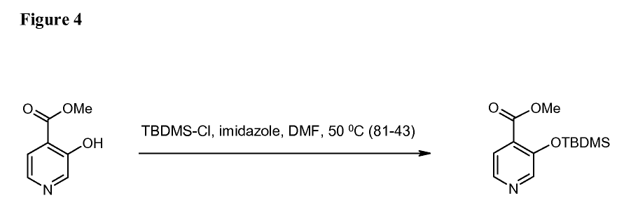

Protection of the 3-OH group is shown in FIG. 4. One of skill in the art will recognize that this is one method among many that could be used.

### Example 4

### Synthesis of 4-(1H-Imidazol-2-yl)-pyridin-3-ol (BST4996)

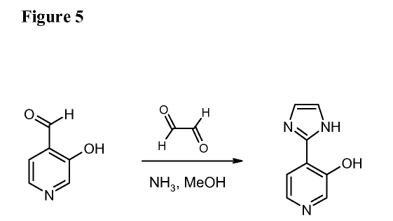

The synthetic scheme for 4-(1H-Imidazol-2-yl)-pyridin-3-ol is provided in FIG. 5. Intermediate compound 3-hydroxy-pyridine-4-carbaldehyde (6 from FIG. 4) (200 mg, 1.63 mmol) was dissolved in MeOH (3 mL) followed by adding glyoxal (40% in water) (1 mL) and NH₄OH (conc. 1 mL). The mixture was stirred for 16 hours with solids formed after about 30 min. Solid was filtered off and MeOH was removed by rotovap. The product was purified by flash column (8:2 EtOAc/MeOH with 1% NH₄OH, R_{f} 0.2) and LC, yield, 176 mg, 1.09 mmol, 67% (LC purity: 100%).

### Physical Properties:

### Compound: C₈H₇N₃O, FW: 161.16

### Purification Method Flash Column

### Purity: 99%

TABLE 3
Spectral Properties

| Compound | | pH 2.0 | pH 7.4 | pH 9.4 |
|---|---|---|---|---|
| BST 4996 | λₘₐₓ₁ (nm) | 287-294 (flat) | 248 | 240 |
| | εₘₐₓ₁ (×10⁻³) | 7.78 | 8.39 | 9.99 |
| | λₘₐₓ₂ (nm) | 323-327 (flat) | 307 | 276 |
| | εₘₐₓ₂ (×10⁻³) | 7.46 | 5.43 | 6.24 |
| | λₘₐₓ₃ (nm) | 371 | 357-367 (flat) | 344 |
| | εₘₐₓ₃ (×10⁻³) | 2.43 | 7.47 | 7.23 |

### Example 5

### Production of Various Other Derivatives

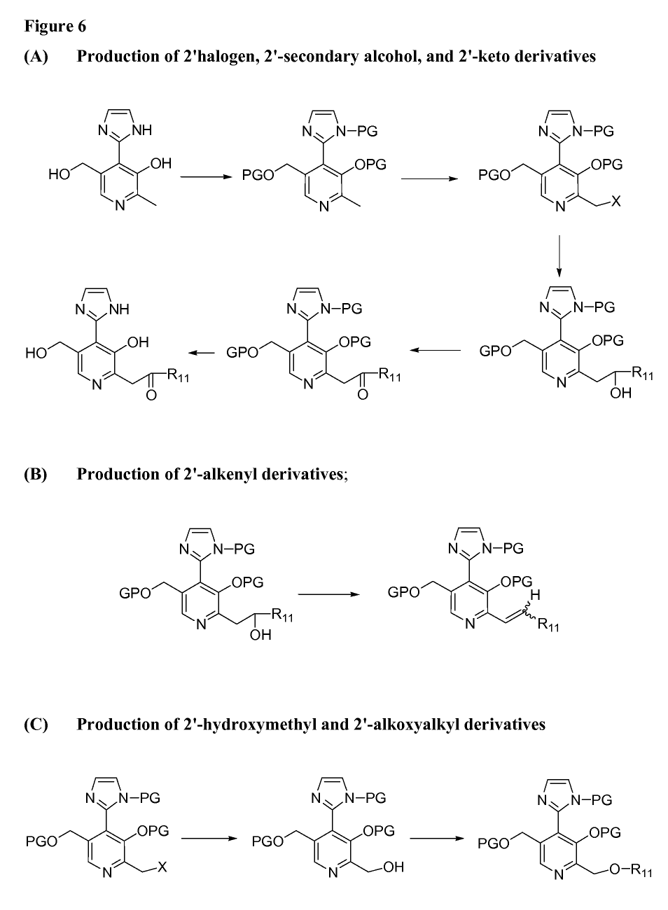

FIG. 6(A)-(E) provide non-limiting examples of synthetic schemes that can be employed to produce other compounds of the invention. “PG” refers to “protecting groups”. The various protecting groups may be the same or different. For example, silyl groups may be used on the oxygens and benzyl groups on the nitrogen, or all protecting groups may comprise benzyl groups.

The dehydration/elimination reaction shown in FIG. 6(B) can be conducted using methods well known in the art.

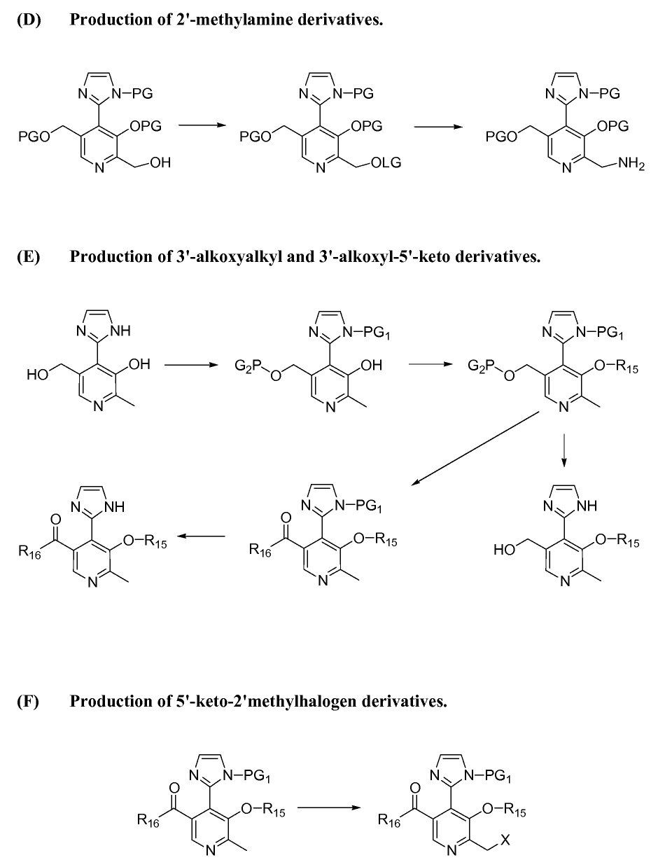

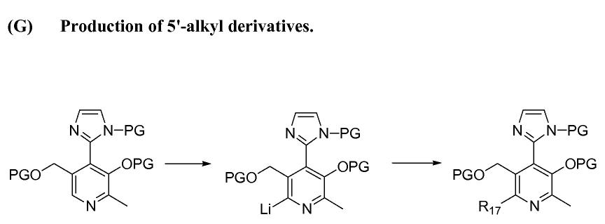

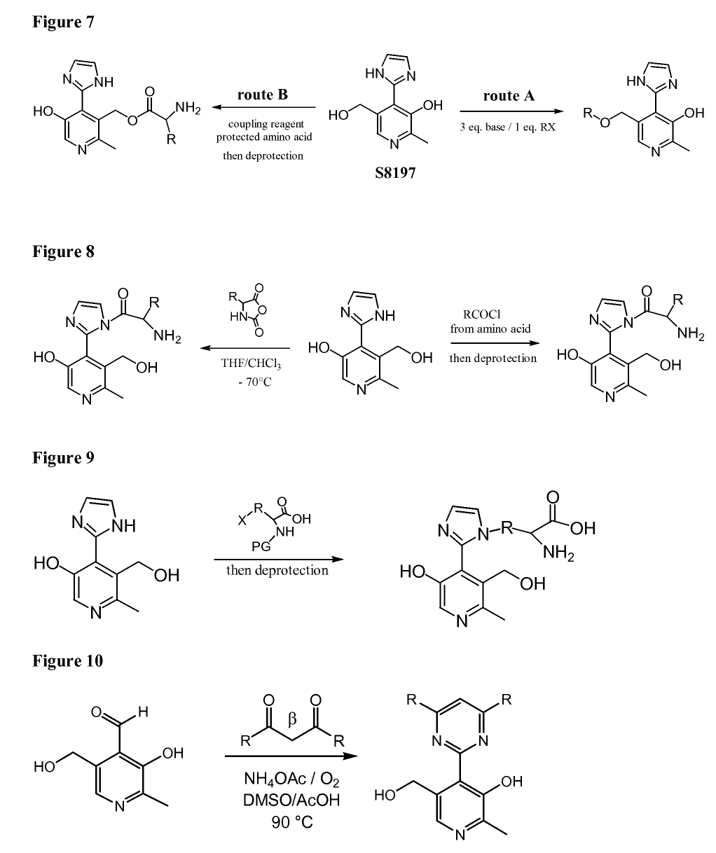

FIG. 7 details one method for modifying the hydroxymethyl group of BST-4997 to provide various derivatives thereof. Such modification is accomplished by introduction of an alkyl group or of any amino acid. This reaction is carried out in one step by reaction of BST-4997 with an alkyl halide (ie: the “X” is a halogen) in the presence of a base as outlined in route A of FIG. 7. The introduction of an amino acid moiety is accomplished using standard coupling reagent such as dicyclocarbonyldiimine in presence of a catalytic amount of dimethylaminopyridine as shown in route B of FIG. 7. These processes are carried out according to methods known in the art.

FIG. 8 details one method for modifying the nitrogen atom in the imidazole ring of BST-4997 to provide various derivatives thereof. Such modification is accomplished by reaction of BST-4997 with an acyl chloride derived from, for example, any amino acid, in apolar solvent. Alternatively, an oxazolidinedione derivative of any amino acid is employed in the presence of an organic base instead of the acyl chloride in the presence of a base such as triethylamine in tetrahydrofuran, chloroform mixture at low temperature. This pathway requires only a one step reaction using, for example, commercially available glycine-derived oxazolidinedione. Oxazolidinediones derived from other amino acids can be synthesized according to the one step Schöllkopf (Synthesis (1981) 966-971) procedure using phosgene or trisphosgene reagent in the presence of base depending on the necessity to protect the amino acid substituents prior to the ring closure reaction. Oxazolidinediones from various amino acids such as arginine are described in literature (J. Am. Chem. Soc. (1971) 93:2746-2754).

FIG. 9 details another method for modifying the nitrogen atom in the imidazole ring of BST-4997 to provide various derivatives thereof. By selection of an appropriate amino acid bearing a good leaving group, such as a halide substituent in the β-position, the corresponding amino acid substituted imidazole is accessible using standard alkylation conditions. In a typical reaction to form amino acid-substituted imidazole derivatives, the imidazole and halide-substituted amino acid are mixed in equal molar portions and the mixture is preferably heated in a polar solvent such as dimethylformamide.

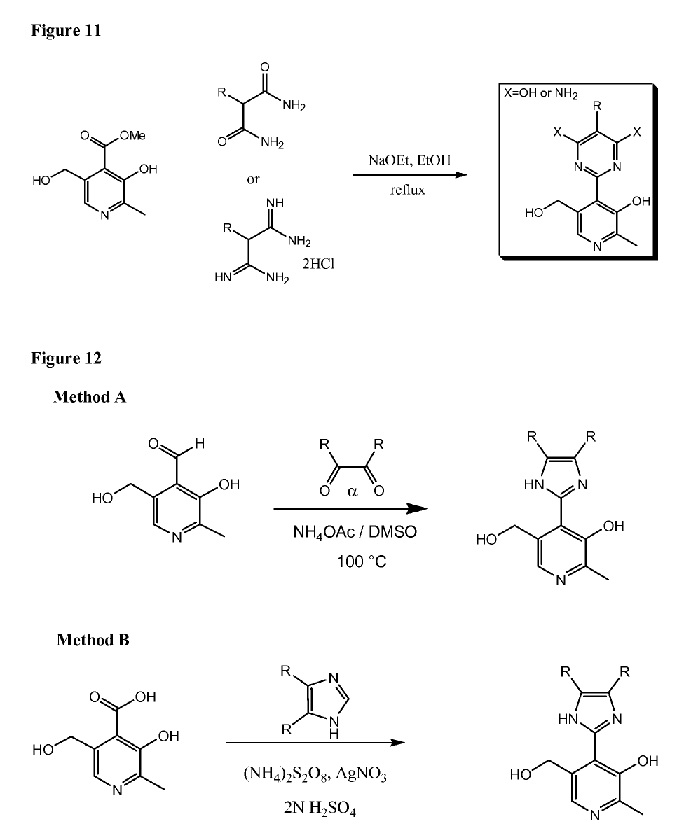

FIG. 10 details one method for forming pyrimidine derivatives using beta diketones. The methods discussed in relation to FIGS. 10 and 11 are based on procedures disclosed by Seko and Rosenbach (Chem. Pham. Bull. (1991) 39(3):651-657; Tetrahedron Letters (1981) 22(15):1453-1454). Pyrimidine derivatives are prepared by reaction of the pyridoxal hydrochloric with 1,3 diketone as outlined in FIG. 10. The reactions occurs when 1,3-diketones are treated with the pyridoxal and ammonium salt in polar solvent, such as dimethylsulfoxyde (DMSO)/acetic acid (AcOH), under oxidating (O₂) conditions over several hours. Under these conditions, pyrimidine derivatives are easily purified by column chromatography on silica gels.

FIG. 11 details another method for forming pyrimidine derivatives. Malonamides or malonimidamides are reacted with esters or other activated carboxylic acid derivatives under basic conditions, as outlined in FIG. 11 (J. Chem. Soc. (1951), 2214; J. Chem. Soc. (1956), 2312; J. Chem. Soc. (1943), 574). Using this method, other functionalities can be introduced, such as hydrophilic substituents including amino or hydroxyl groups, as shown in the figure.

FIG. 12 details two methods for forming imidazole derivatives. Method A involves the reaction of pyridoxal hydrochloride and 1,2-diketones under essentially the same conditions as those described above for reaction with 1,3 diketones. Method B utilizes an available 4-pyridoxic acid according to a reaction described by Pellicciari with imidazole compounds (Arzneim. Forsch (1980) 30:2103-2105). This approach is based on silver catalyzed decarboxylation of carboxylic acid in methanol-water by peroxydisulfate, followed by reaction of the radical formed with imidazoles.

Using the various methods disclosed above, either alone or in combination with further methods known to those in the art, a large variety of compounds according to the present invention can be prepared. Other specific embodiments of the compounds of the invention include the following:

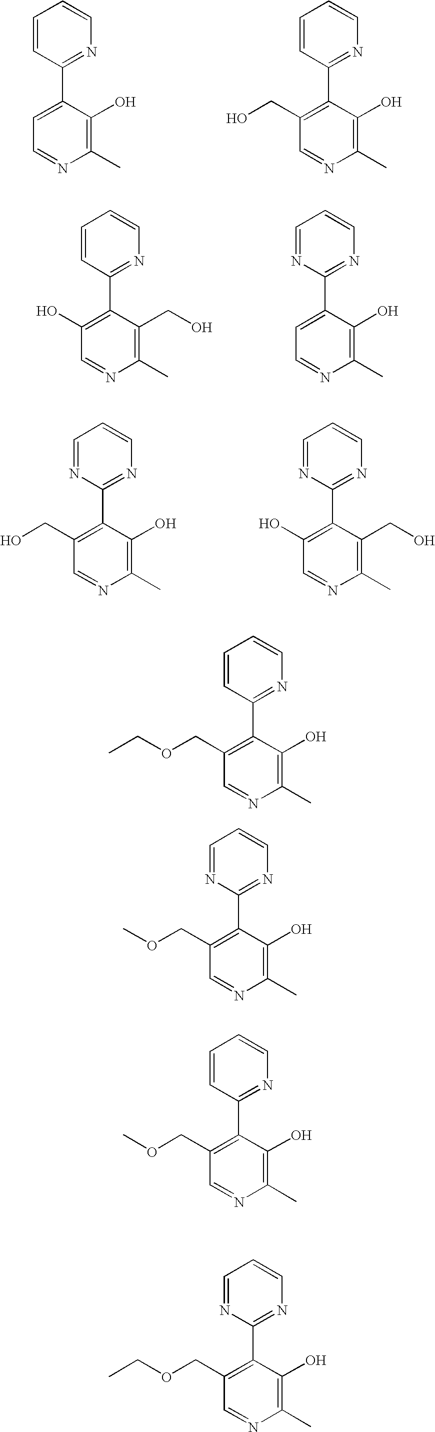

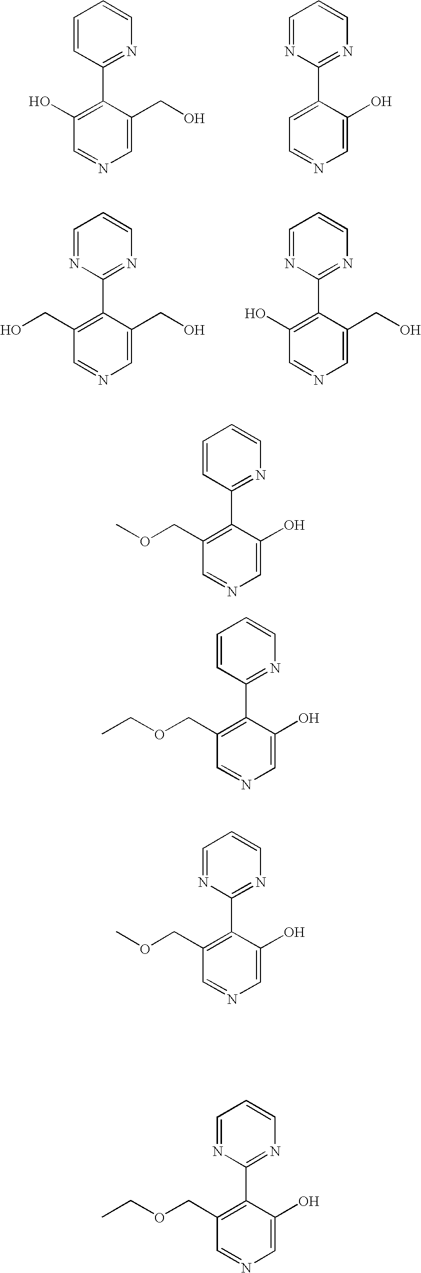

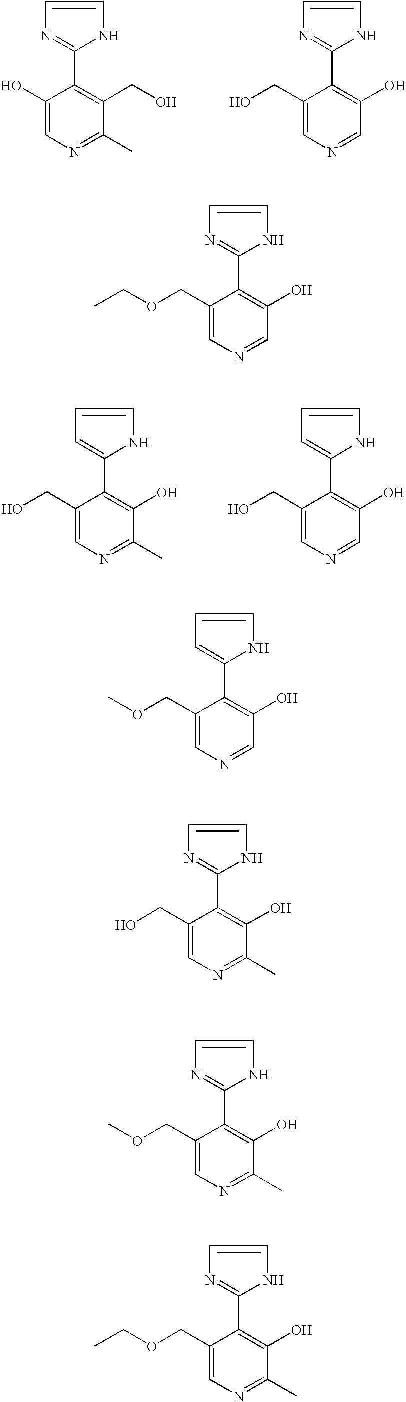

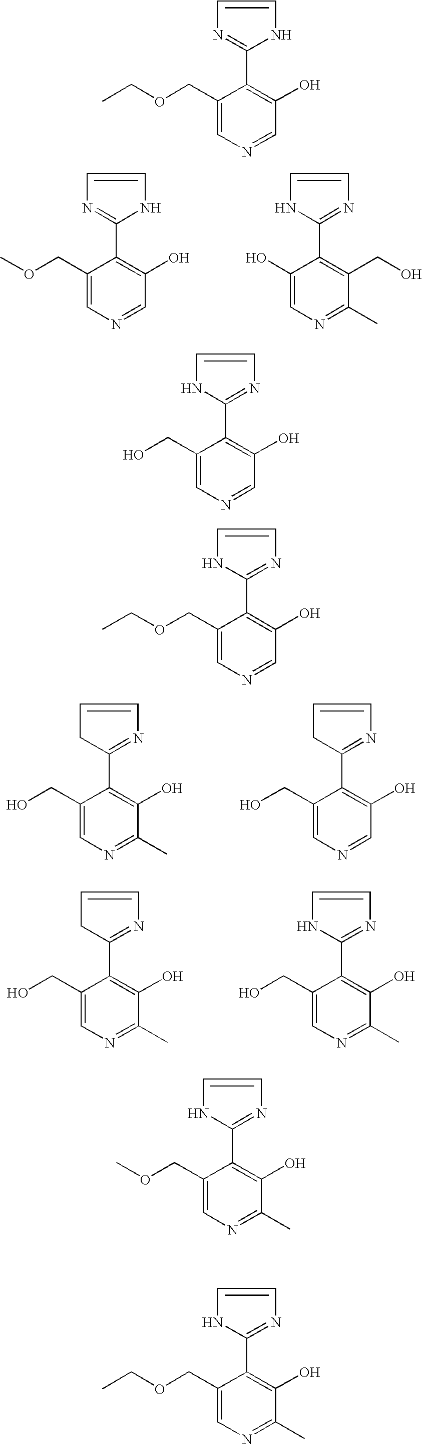

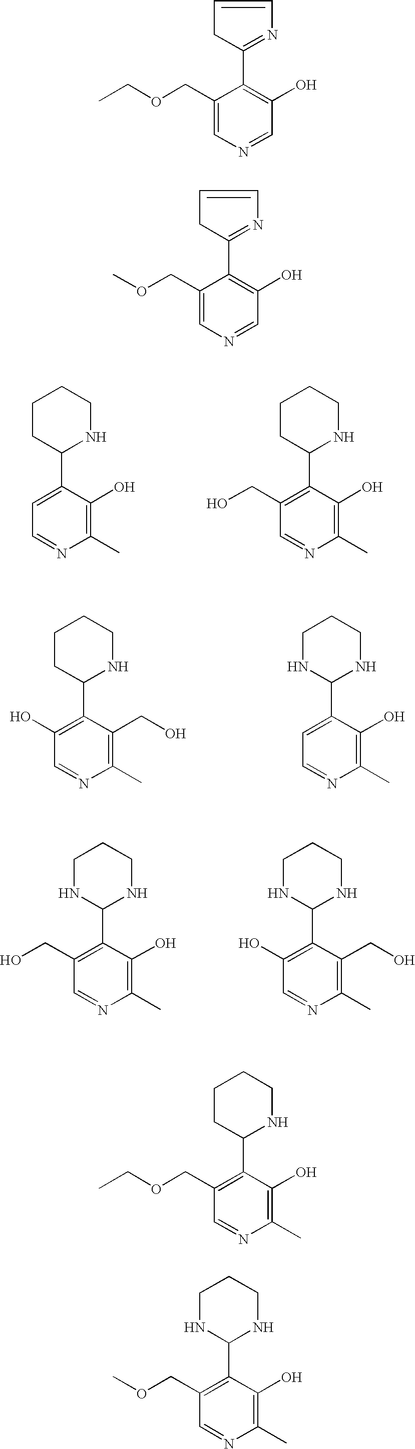

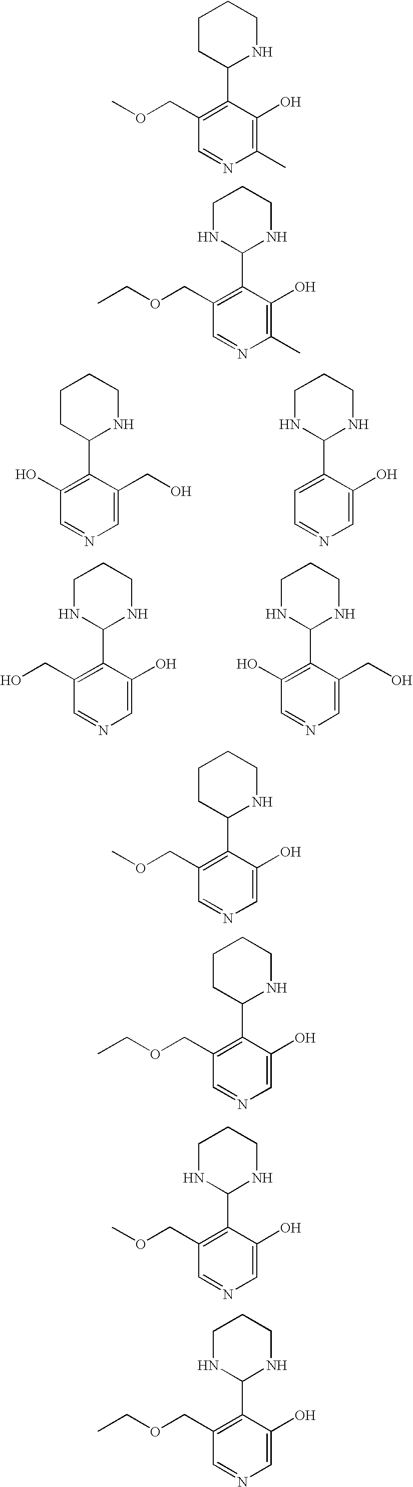

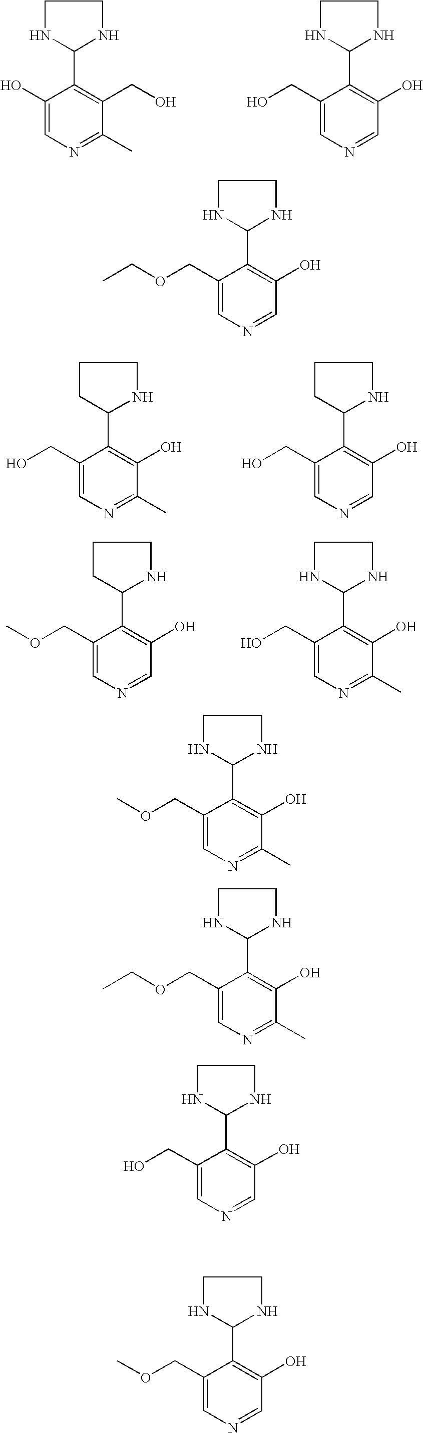

### Example 6

### In Vitro Method to Identify Inhibitors of Post-Amadori AGE Formation

The effect of [2,4′]Bipyridinyl-3′-ol, 5-Hydroxymethyl-4-(1H-imidazol-2-yl)-2-methyl-pyridin-3-ol, and 4-(1H-Imidazol-2-yl)-pyridin-3-ol on post-Amadori AGE formation during interrupted glycation of bovine serum albumin and ribonuclease A by ribose was determined in comparison to pyridoxamine. Modification with ribose was done at 37° C. in 0.2 M phosphate buffer of pH 7.5 containing 0.02% sodium azide. The solutions were kept in capped tubes and opened only to remove timed aliquots that were immediately frozen for later carrying out the various analyses. “Interrupted glycation” experiments were carried out by first incubating protein with the ribose at 37° C. for 8 or 24 h, followed by immediate and extensive dialysis against frequent cold buffer changes at 4° C. The samples were then re-incubated by quickly warming to 37° C. in the absence of external ribose. Aliquots were taken and frozen at various intervals for later analysis. (See U.S. Pat. No. 5,985,857)

The interrupted glycation method for following post-Amadori kinetics of AGE formation allows for the rapid quantitative study of “late” stages of the glycation reaction. Importantly, this method allows for inhibition studies that are free of pathways of AGE formation that arise from glycoxidative products of free sugar or Schiff base (Namiki pathway). The experiments were designed to determine the half-maximal inhibitory concentration of these compounds (“IC50 values”) for inhibiting the conversion of Amadori compounds to post Amadori advanced glycation endproducts.

The IC50 values determined were as follows:
[2,4′]Bipyridinyl-3′-ol (BST4944): 2 mM
5-Hydroxymethyl-4-(1H-imidazol-2-yl)-2-methyl-pyridin-3-ol (BST4997): 0.2-0.3 mM
4-(1H-Imidazol-2-yl)-pyridin-3-ol (BST4996): 0.2-0.3 mM

These data demonstrate that these 3 compounds were very effective at inhibiting the conversion of Amadori compounds to post Amadori advanced glycation endproducts. In comparison, pyridoxamine inhibited the conversion of Amadori compounds to post Amadori advanced glycation endproducts with a half-maximal inhibitory concentration of approximately 3 mM.

### Example 7

### Effect of 5-Hydroxymethyl-4-(1H-imidazol-2-yl)-2-methyl-pyridin-3-ol (BST-4997) in rat model of diabetic neuropathy

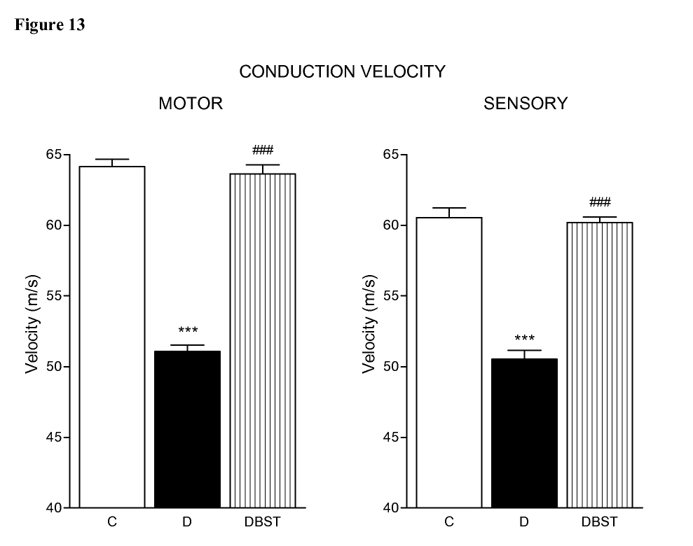

Our aim was to ascertain whether BST-4997 treatment could correct nerve dysfunction in streptozotocin (STZ)-diabetic rats. Animals (n=10) were made diabetic for 6 weeks (group D), following which they were treated for 2 weeks with BST-4997 (group DBST) given in the drinking water at a concentration of 50 mg/L. They were compared to control non-diabetic rats (groups C). The data are presented in FIG. 13-15. Statistical significance is reported according to the legend: **, *** p<0.01, p<0.001 versus untreated controls; ### p<0.001 treatment effect versus untreated diabetics.

FIG. 13 provides a graphical representation of the effect of BST-4997 on restoring nerve conduction velocity (NCV) in the STZ rats. Motor NCV was tested between the sciatic notch and knee for the nerve branch to tibialis anterior muscle, as described in Cameron et al., Q. J. Exp. Physiol. 74: 917-926 (1989); and Cameron et al., Exp. Neurol. 92: 757-761 (1986). Saphenous sensory NCV was measured between the groin and ankle.

These data clearly demonstrate that BST-4997 dramatically reduced the diabetes-associated defect in both motor and sensory NCV in the STZ rats.

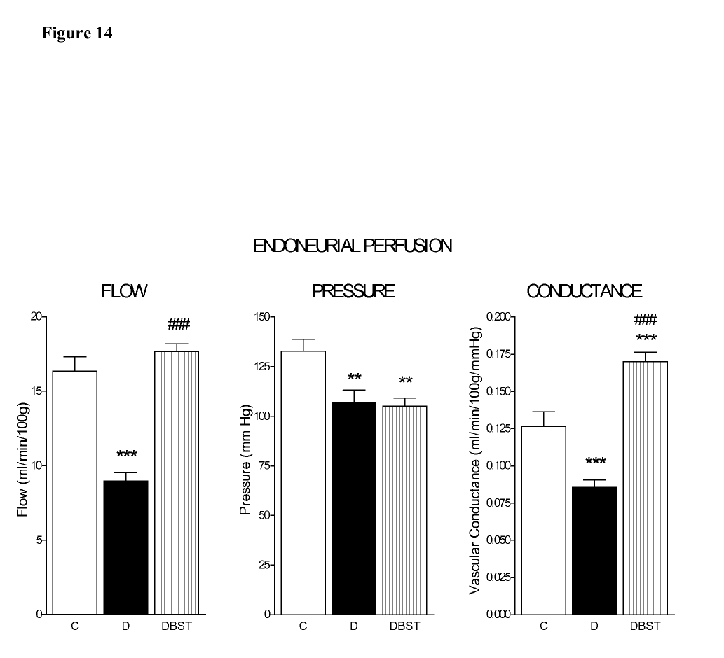

FIG. 14 provides a graphical representation of the effect of BST-4997 on restoring endoneurial perfusion in the STZ rats. Vascular blood flow, pressure, and conductance were measured. These data clearly demonstrate that BST-4997 dramatically reduced the diabetes-associated defects in endoneurial blood flow and conductance.

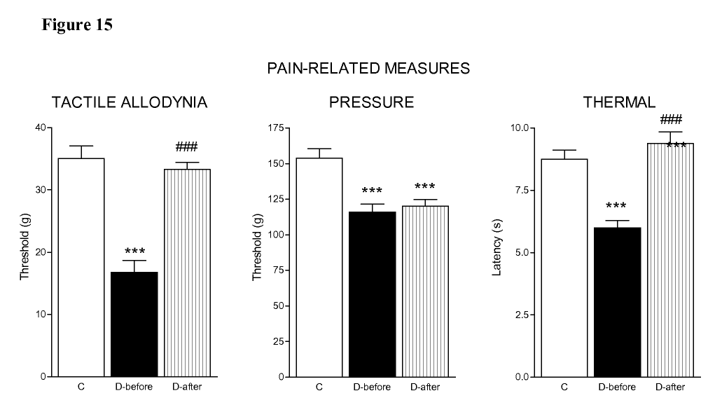

FIG. 15 provides a graphical representation of the effect of BST-4997 on improving pain related measures in the STZ rats. Responses to tactile allodynia, pressure, and thermal stimuli were measured. These data clearly demonstrate that BST-4997 dramatically reduced the diabetes-associated defects in tactile allodynia and thermal hyperalgesia, as measured by latency for foot withdrawal from a noxious heat stimulus.

Thus, these data clearly demonstrate that BST-4997 is effective in correcting nerve dysfunction in a state of the art rat model of diabetic nephropathy.

### Example 8

### Compound Binding to Plasma Albumin

This study was done to measure the binding of compounds of the present invention to plasma albumin. Such binding can result in longer plasma retention times and enhanced therapeutic efficacy for the compounds. The proteins tested were bovine serum albumin, bovine (BSA) at 40 mg/ml and rat albumin at ˜13 mg/ml.

Spectrophotmetry: Two mL of protein solution in 0.1M phosphate buffer saline (PBS) at pH 7.4 was titrated with compound in 0.2 M phosphate buffer at pH 7.4. The protein concentration was prepared to have absorbance less than 1 at the observation wavelength range (250-500), and the total volume change during titration was controlled below 2%. The compound-protein binding detection was based on the shift of spectrum.

Free compound measurement: One ml of compound and protein mixture was incubated at 37° C. for about 30 min and loaded into Centricon YM-10 (10,000 MW cut-off, for BSA) or Microcon YC-3 (3,000 MW cut-off). The samples were centrifuged in a fixed-angle rotor at 6,000 rpm for about 6-8 min (Centricon) or at 9,000 rpm for about 15 min (Microcon) to allow about 10-20% of the volume to filter through. Free compound passes through the membrane while the free and complexed protein remains in the sample reservoir. The concentration of free compound in the filtrate is assumed to be the same as in the sample above the membrane. A fixed amount of filtrate aliquot was diluted to 0.6 ml with PBS at pH 7.4 (PBS was prepared using 1 tablet from Sigma dissolved in 200 ml deionized H₂O) and measured by UV absorbance. To quantify the percentage of free compound, the same compound at the same concentration in PBS solution, as standard, was prepared in parallel through the same process, or a calibration curve was generated through the same process. 1 ml of PBS solution (as blank) and 1 ml of protein solution (as control) also went through the same process and measurement.

Calculations: The method used to determine the percentage of free compound was either (a) % Free compound=(Aₛₐₘₚₗₑ−A_{control})/A_{standard}×100, where, A is absorbance at selected wavelength; or (b) Obtaining free compound concentration of sample from calibration curve, and then divided by total concentration: K_{d}=[P][L]/[PL] (for 1:1 binding), using BSA MW=70,000.

Results: The results are provided in Tables 4-5 below, and demonstrate that the compounds tested all bind to albumin, which may provide for enhanced plasma retention times and efficacy of the compounds.

TABLE 4
Results of BST4997 study

| | | | Computed |
|---|---|---|---|
| BST4997 | | % Free | (1:1) |
| Conc. | Protein | BST4997 | K_{d} (mM) |
| 10 μg/ml | rat albumin ~13 mg/ml | 67% | 0.35 |
| 5 μg/ml | BSA | 31% | 0.26 |
| | 40 mg/ml | | |
| 10 μg/ml | BSA | 30% | 0.24 |
| | 40 mg/ml | | |
| 20 μg/ml | BSA | 38% | 0.32 |
| | 40 mg/ml | | |

TABLE 5
Results of BST-4996 and BST-4944 studies

| | Compd. | | % Free |
|---|---|---|---|
| Compound | Conc. | Protein | Compound |
| BST4996 | 8 μg/ml | BSA 40 mg/ml | 53% |
| BST4944 | 8 μg/ml | BSA 40 mg/ml | 34% |

I claim:

1. A compound of general formula I: 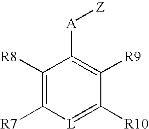
or pharmaceutically acceptable salts thereof, wherein
L is N, N⁺O⁻, or N⁺—Z with any counterion, wherein Z is C₁-C₆ alkyl;
A is a bond, C₁-C₄ alkyl, —O—C₁-C₄ alkyl, —C₁-C₄ alkyl-O—, C₁-C₄ alkoxy C₁-C₄ alkyl-, —N(R₂₀)C₁-C₄ alkyl, —C₁-C₄ alkyl-N(R₂₀)—, —C₁-C₂ alkyl-N(R₂₀)—C₁-C₂ alkyl, —S—C₁-C₄ alkyl, —C₁-C₄ alkyl-S—, or C₁-C₄ thioalkoxy C₁-C₄ alkyl-, wherein R₂₀ is H or C₁-C₄ alkyl;
R₈ is H, —CH₂OR₂ or OR₂;
R₉ is —CH₂OR₁ or OR₁;
R₁ and R₂ are independently H, C₁-C₆ alkyl, C₁-C₆ alkanoyl, C(O)NR₃R₄, C₁-C₆ alkoxy C₁-C₆ alkyl, arylalkyl or arylalkanoyl, wherein
the alkyl, alkanoyl and alkoxy groups are unsubstituted or substituted with 1, 2, or 3 groups that are independently hydroxy, C₁-C₄ alkoxy or NH₂;
R₃ and R₄ are independently H, C₁-C₆ alkyl, C₁-C₆ alkoxy, arylalkyl, arylalkanoyl, or —CO₂alkyl, —CO₂alkylaryl; wherein
the aryl portion of each arylalkyl or each arylalkanoyl is unsubstituted or substituted with 1, 2, 3, 4, 5 groups that are independently C₁-C₄ alkyl, C₁-C₄ alkoxy, hydroxy, halogen, haloalkyl, haloalkoxy, or nitro;
n is 0, 1, 2, or 3;
R₇ and R₁₀ are independently H, C₁-C₆ alkyl or C₂-C₈ alkenyl, each of which is unsubstituted or substituted by 1 or 2 groups that are independently hydroxy, halogen, NR₃R₄, alkoxy, heteroarylalkoxy, heterocycloalkylalkoxy, arylalkoxy, or aryl; wherein 1 or 2 carbons of the alkyl or alkenyl group can be replaced with a C(O) group or a CHO group;
Z is heterocycloalkyl or heteroaryl, which is unsubstituted or substituted with 1, 2, 3, or 4 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, C₁-C₆ alkanoyl, hydroxy, hydroxy C₁-C₆ alkyl, NR₃R₄, or —C₁-C₆ alkyl NR₃R₄, wherein
R₃ and R₄ are independently H, C₁-C₆ alkyl, C₁-C₆ alkoxy, arylalkyl, arylalkanoyl, —CO₂alkyl, or —CO₂ alkylaryl;
each alkyl, alkoxy, and alkanoyl group is unsubstituted or substituted with 1, 2, or 3, groups that are independently hydroxy or halogen,
the aryl, heteroaryl, and heterocycloalkyl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro;
any NH group in a heterocycloalkyl or heteroaryl group can optionally be NR₃, where R₃ is defined above;
provided that the Z group is attached to the CH₂ group or the pyridine ring through a carbon-carbon bond;
provided that when Z is tetrahydropyridine or piperidine, the Z group is attached via a carbon that is adjacent to a nitrogen atom.

2. The compound of claim 1, wherein the Z group contains at least one nitrogen atom.

3. The compound of claim 1 wherein
A is C₁-C₄ alkyl, —O—C₁-C₄ alkyl, —C₁-C₄ alkyl-O—, C₁-C₄ alkoxy C₁-C₄ alkyl-, —N(R₂₀)C₁-C₄ alkyl, —C₁-C₄ alkyl-N(R₂₀)—, —C₁-C₂ alkyl-N(R₂₀)—C₁-C₂ alkyl, —S—C₁-C₄ alkyl, —C₁-C₄ alkyl-S—, or C₁-C₄ thioalkoxy C₁-C₄ alkyl-, wherein R₂₀ is H or C₁-C₄ alkyl;
R₇ and R₁₀ are independently H, C₁-C₆ alkyl or C₂-C₈ alkenyl, each of which is unsubstituted or substituted by 1 or 2 groups that are independently hydroxy, NR₃R₄, heteroarylalkoxy, heterocycloalkylalkoxy, arylalkoxy, or aryl; wherein 1 or 2 carbons of the alkyl or alkenyl group can be replaced with a C(O) group or a CHO group; and
Z is quinazoline; quinoxaline; imidazole; benzimidazole; piperazine; morpholine; thiomorpholine; quinoline; isoquinoline; 3-, 4-, 5-, 6-, 7-, or 8-tetrahydroisoquinoline; 1,2,4-triazole; hexahydropyridazine; tetrahydropyridazine; pyrazole; pyrrole; pyrimidine; pyrazine; isothiazole; 4(3H)-pyrimidinone; isoxazole; 1,3,5-triazine; hexahydropyrimidine; furan; tetrahydrofuran; tetrahydropyrimidine; piperidine; tetrahydropyridine; indole; indoline; benzoxazole; 1H-1,2,3-triazole; azocine; or imidazolidine; each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, C₁-C₆ alkanoyl, hydroxy, hydroxy C₁-C₆ alkyl, NR₃R₄, or —C₁-C₆ alkyl NR₃R₄, wherein R₃ and R₄ at each occurrence areas defined above and
each alkyl, alkoxy, and alkanoyl group is unsubstituted or substituted with 1, 2, or 3, groups that are independently hydroxy or halogen,
the aryl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro,
any NH group in a heterocycloalkyl or heteroaryl group can optionally be NR₃, where R₃ is defined above;
provided that the Z group is attached to the CH₂ group through a carbon-carbon bond;
provided that when Z is tetrahydropyridine or piperidine, the Z group is attached via a carbon that is adjacent to a nitrogen atom.

4. The compound of claim 3 wherein Z contains at least two nitrogen atoms.

5. The compound of claim 4 wherein
R₇ and R₁₀ are independently H, C₁-C₆ alkyl, which is unsubstituted or substituted by 1 or 2 groups that are independently hydroxy, NR₃R₄, heteroarylalkoxy, heterocycloalkylalkoxy, arylalkoxy, or aryl; and
Z is quinazoline; quinoxaline; imidazole; benzimidazole; piperazine; 1,2,4-triazole; hexahydropyridazine; tetrahydropyridazine; pyrazole; pyrimidine; pyrazine; 4(3H)-pyrimidinone; 1,3,5-triazine; hexahydropyrimidine; tetrahydropyrimidine; tetrahydropyridine; indole; indoline; 1H-1,2,3-triazole; or imidazolidine, each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, alkanoyl, hydroxy C₁-C₆ alkyl, NR₃R₄, or —C₁-C₆ alkyl-NR₃R₄, wherein
R₃ and R₄ at each occurrence are independently H, C₁-C₆ alkyl, C₁-C₆ alkoxy, arylalkyl, arylalkanoyl, C₁-C₆ alkanoyl, —CO₂alkyl, —CO₂alkylaryl
each alkyl, alkoxy, and alkanoyl group is unsubstituted or substituted with 1, 2, or 3, groups that are independently hydroxy or halogen,
the aryl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro,
any NH group in a heterocycloalkyl or heteroaryl group can optionally be NR₃, where R₃ is defined above; and
provided that the Z group is attached to the CH₂ group or the pyridine ring through a carbon-carbon bond.

6. The compound of claim 5 wherein
R₇ and R₁₀ are independently H, C₁-C₆ alkyl which is unsubstituted or substituted by 1 or 2 groups that are independently hydroxy, or NR₃R₄; wherein 1 or 2 carbons of the alkyl or alkenyl group can be replaced with a C(O) group or a CHO group; and
Z is imidazole; benzimidazole; piperazine; 1,2,4-triazole; hexahydropyridazine; tetrahydropyridazine; pyrazole; pyrimidine; pyrazine; hexahydropyrimidine; tetrahydropyrimidine; tetrahydropyridine; indole; indoline; 1H-1,2,3-triazole; or imidazolidine, each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, phenyl C₁-C₆ alkyl, phenyl C₁-C₆ alkanoyl, phenyl C₁-C₆ alkoxy, hydroxy C₁-C₆ alkyl, NR₃R₄, or —C₁-C₆ alkyl-NR₃R₄, wherein
R₃ and R₄ are independently H, C₁-C₆ alkyl, benzyl, benzoyl, C₁-C₆ alkanoyl, —CO₂alkyl, —CO₂alkylphenyl;
each alkyl, alkoxy, and alkanoyl group is unsubstituted or substituted with 1, 2, or 3, groups that are independently hydroxy, fluoro, or chloro;
the phenyl groups are unsubstituted or substituted with 1, 2, 3, 4, or 5 groups that are C₁-C₄ alkyl, C₁-C₄ alkoxy, hydroxy, fluoro, chloro, CF₃, OCF₃, or nitro;
any NH group in a heterocycloalkyl or heteroaryl group can optionally be NR₃, where R₃ is defined above;
provided that the Z group is attached to the CH₂ group through a carbon-carbon bond;
provided that when Z is tetrahydropyridine or piperidine, the Z group is attached via a carbon that is adjacent to a nitrogen atom.

7. The compound of claim 3 wherein
the aryl portion of each arylalkyl or each arylalkanoyl is unsubstituted or substituted with 1, 2, 3, 4, or 5 groups that are independently C₁-C₄ alkyl, C₁-C₄ alkoxy, hydroxy, halogen, haloalkyl, haloalkoxy, or nitro; and
the alkyl, alkanoyl and alkoxy groups are independently substituted with 1, 2, or 3 groups that are independently hydroxy, C₁-C₄ alkoxy or NH₂.

8. The compound of claim 6 wherein
R₁ and R₂ are independently hydrogen, C₁-C₄ alkyl, or benzyl;
wherein the phenyl portion of each benzyl is unsubstituted or substituted with 1, 2, or 3, groups that are independently C₁-C₄ alkyl, C₁-C₄ alkoxy, hydroxy, fluoro, chloro, CF₃, OCF₃, or nitro; and
wherein the alkyl groups are independently substituted with 1, or 2, groups that are independently hydroxy, methoxy, ethoxy, propoxy, isopropoxy or NH₂.

9. The compound of claim 4 wherein
R₇ and R₁₀ are independently H, C₁-C₆ alkyl, which is unsubstituted or substituted by 1 or 2 groups that are independently hydroxy, or NR₃R₄, heteroarylalkoxy, heterocycloalkylalkoxy, arylalkoxy, or aryl; and
the aryl, heteroaryl, or heterocycloalkyl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro,

10. The compound of claim 1 wherein the compound is a compound of the formula:

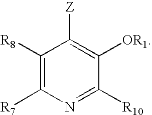

11. The compound of claim 10 wherein the Z group contains at least one nitrogen atom.

12. The compound of claim 11 wherein
Z is quinazoline; quinoxaline; imidazole; benzimidazole; piperazine; morpholine; thiomorpholine; quinoline; isoquinoline; 3, 4, 5, 6, 7, or 8-tetrahydroisoquinoline; 1,2,4-triazole; hexahydropyridazine; tetrahydropyridazine; pyrazole; pyrimidine; pyrazine; isothiazole; 4(3H)-pyrimidinone; isoxazole; 1,3,5-triazine; hexahydropyrimidine; furan; tetrahydrofuran; tetrahydropyrimidine; piperidine; tetrahydropyridine; indole; indoline, benzoxazole; 1H-1,2,3-triazole; azocine; or imidazolidine; each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo C₁-C₆ alkyl, halo C₁-C₆ alkyl, aryl C₁-C₆ alkyl, aryl C₁-C₆ alkanoyl, aryl C₁-C₆ alkoxy, alkanoyl, hydroxy C₁-C₆ alkyl, NR₃R₄, or —C₁-C₆ alkyl NR₃R₄, wherein
R₃ and R₄ are independently H, C₁-C₆ alkyl, C₁-C₆ alkoxy, arylalkyl, arylalkanoyl, C₁-C₆ alkanoyl, —CO₂alkyl, —CO₂alkylaryl;
the aryl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro,
provided that the Z group is attached to the pyridine ring through a carbon-carbon bond; and
provided that when Z is hexahydropyrimidine, it is substituted with two or three groups.

13. The compound of claim 12 wherein Z contains at least two nitrogen atoms.

14. The compound of claim 13 wherein
Z is quinazoline; quinoxaline; imidazole; benzimidazole; piperazine; 1,2,4-triazole; hexahydropyridazine; tetrahydropyridazine; pyrazole; pyrimidine; pyrazine; 4(3H)-pyrimidinone; 1,3,5-triazine; hexahydropyrimidine; tetrahydropyrimidine; tetrahydropyridine; indole; indoline; 1H-1,2,3-triazole; or imidazolidine, each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, alkanoyl, hydroxy C₁-C₆ alkyl, NR₃R₄, or —C₁-C₆ alkyl-NR₃R₄.

15. The compound of claim 14 wherein
Z is imidazole; benzimidazole; piperazine; 1,2,4-triazole; hexahydropyridazine; tetrahydropyridazine; pyrazole; pyrimidine; pyrazine; hexahydropyrimidine; tetrahydropyrimidine; tetrahydropyridine; indole; indoline; 1H-1,2,3-triazole; or imidazolidine, each of which is unsubstituted or substituted with 1, 2, or 3 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, phenyl C₁-C₆ alkyl, phenyl C₁-C₆ alkanoyl, phenyl C₁-C₆ alkoxy, hydroxy C₁-C₆ alkyl, NR₃R₄, or —C₁-C₆ alkyl-NR₃R₄, wherein
R₃ and R₄ are independently H, C₁-C₆ alkyl, benzyl, benzoyl, C₁-C₆ alkanoyl, —CO₂alkyl, —CO₂ alkylphenyl;
each alkyl, alkoxy, and alkanoyl group is unsubstituted or substituted with 1, 2, or 3, groups that are independently hydroxy, fluoro, or chloro; and
the phenyl groups are unsubstituted or substituted with 1, 2, 3, 4, or 5 groups that are C₁-C₄ alkyl, C₁-C₄ alkoxy, hydroxy, fluoro, chloro, CF₃, OCF₃, or nitro.

16. The compound of claim 15 wherein
R₁ and R₂ are independently H, C₁-C₆ alkoxy C₁-C₆ alkyl, arylalkyl or arylalkanoyl, wherein and
the alkyl, alkanoyl and alkoxy groups are independently substituted with 1, 2, or 3 groups that are independently hydroxy, C₁-C₄ alkoxy or NH₂.

17. The compound of claim 16 wherein
R₁ and R₂ are independently hydrogen, C₁-C₄ alkyl, or benzyl, wherein
the phenyl portion of each benzyl is unsubstituted or substituted with 1, 2, or 3, groups that are independently C₁-C₄ alkyl, C₁-C₄ alkoxy, hydroxy, fluoro, chloro, CF₃, OCF₃, or nitro; and
the alkyl groups are independently substituted with 1, or 2, groups that are independently hydroxy, methoxy, ethoxy, propoxy, isopropoxy or NH₂.

18. The compound of claim 1 wherein the compound is a compound of the formula: 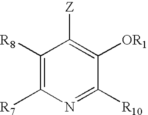
or pharmaceutically acceptable salts thereof, and wherein
R₈ is H, —CH₂OR₂ or OR₂;
R₁ and R₂ are independently H, C₁-C₆ alkyl, C₁-C₆ alkanoyl, C₁-C₆ alkoxy C₁-C₆ alkyl, arylalkyl or arylalkanoyl, wherein
the alkyl, alkanoyl and alkoxy groups are unsubstituted or substituted with 1, 2, or 3 groups that are independently hydroxy, C₁-C₄ alkoxy or NH₂;
R₃ and R₄ are independently H, C₁-C₆ alkyl, C₁-C₆ alkoxy, arylalkyl, arylalkanoyl, or —CO₂alkyl, —CO₂alkylaryl; wherein
the aryl portion of each arylalkyl or each arylalkanoyl is unsubstituted or substituted with 1, 2, 3, 4, 5 groups that are independently C₁-C₄ alkyl, C₁-C₄ alkoxy, hydroxy, halogen, haloalkyl, haloalkoxy, or nitro;
R₇ and R₁₀ are independently H, C₁-C₆ alkyl or C₂-C₈ alkenyl, each of which is unsubstituted or substituted by 1 or 2 groups that are independently hydroxy, halogen, NR₃R₄, alkoxy, heteroarylalkoxy, heterocycloalkylalkoxy, arylalkoxy, or aryl; wherein 1 or 2 carbons of the alkyl or alkenyl group can be replaced with a C(O) group or a CHO group;
Z is heterocycloalkyl or heteroaryl containing 1, 2, or 3 nitrogen atoms, which is unsubstituted or substituted with 1, 2, 3, or 4 groups that are independently C₁-C₆ alkyl, C₁-C₆ alkoxy, C₁-C₆ alkoxy C₁-C₆ alkyl, C₁-C₆ alkoxy C₁-C₆ alkoxy, halo, halo C₁-C₆ alkyl, aryl C₁-C₆ alkyl, aryl C₁-C₆ alkanoyl, aryl C₁-C₆ alkoxy, C₁-C₆ alkanoyl, hydroxy, hydroxy C₁-C₆ alkyl, NR₃R₄, or —C₁-C₆ alkyl NR₃R₄, wherein R₃ and R₄ are as defined above;
each alkyl, alkoxy, and alkanoyl group is unsubstituted or substituted with 1, 2, or 3, groups that are independently hydroxy or halogen,
the aryl, heteroaryl, and heterocycloalkyl groups are unsubstituted or substituted with 1, 2, 3, 4, or 5 groups that are C₁-C₄ alkyl, C₁-C₄ alkoxy, hydroxy, halogen, haloalkyl, haloalkoxy, or nitro;
any NH group in a heterocycloalkyl or heteroaryl group can optionally be NR₃, where R₃ is defined above.

19. The compound of claim 18, wherein Z is heteroaryl.

20. The compound of claim 19, wherein the heteroaryl is selected from the group consisting of pyridine, imidazole, diazole, and triazole.

21. The compound of claim 18 wherein the Z group is attached to the pyridine ring through a carbon-carbon bond.

22. The compound of claim 18 wherein Z contains 2 nitrogen atoms.

23. The compound of claim 21 wherein Z contains an unsubstituted nitrogen atom on both sides of the attachment point of Z.

24. The compound of claim 18 wherein the aryl substituents on Z are phenyl or phenyl derivatives.

25. The compound of claim 1 selected from the group consisting of [2,4′]Bipyridinyl-3′-ol, 4-(1H-Imidazol-2-yl)-pyridin-3-ol, and 5-Hydroxymethyl-4-(1H-imidazol-2-yl)-2-methyl-pyridin-3-ol.

26. A pharmaceutical composition comprising the compound of claim 1 and a pharmaceutically acceptable carrier.

27. A method for treating or inhibiting development of one or more AGE- and/or ALE-associated complications in subject in need thereof comprising administering one or more compounds according to claim 1 to the subject.

28. A method for treating or inhibiting development of one or more AGE- and/or ALE-associated complications in a subject in need thereof comprising administering one or more pharmaceutical compositions according to claim 26 to the subject.

29. The method of claim 27 wherein the one or more AGE- and/or ALE-associated complications are selected from the group consisting of accelerated protein aging, retinopathy, nephropathy, proteinuria, impaired glomerular clearance, neuropathy, hyperlipidemia, hypertriglyceridemia, hypercholesterolemia, atherosclerosis, cardiovascular disease, neurodegenerative amyloid diseases, diabetes-associated hyperlipidemia, oxidative modification of proteins, arthritis, connective tissue diseases, amyloidosis, urinary stone disease, obesity-related complications, proliferation of smooth muscle cells in the aorta, coronary artery occlusion, hypertension; and dialysis-related disorders selected from the group consisting of dialysis-related cardiac morbidity and mortality, dialysis-related amyloidosis, dialysis-related increases in permeability of the peritoneal membrane in a dialysis patient, renal failure progression in a dialysis patient, and ultrafiltration failure and peritoneal membrane destruction in a dialysis patient.

30. A method for treating or inhibiting development of one or more disorders selected from the group consisting of diabetic nephropathy, proteinuria, impaired glomerular clearance, retinopathy, neuropathy, atherosclerosis, diabetes-associated hyperlipidemia, oxidative modification of proteins, arthritis, connective tissue diseases, amyloidosis, urinary stone disease, obesity-related complications proliferation or smooth muscle cells in the aorta, coronary artery occlusion, and hypertension; and dialysis-related disorders including dialysis-related cardiac morbidity and mortality, dialysis-related amyloidosis, dialysis-related increases in permeability of the peritoneal membrane in a dialysis patient, renal failure progression in a dialysis patient, and inhibiting ultrafiltration failure and peritoneal membrane destruction in a dialysis patient, wherein the method comprises administering an effective amount of a compound according to claim 1 to a subject in need of such treatment.

31. A method for treating or inhibiting development of one or more disorders selected from the group consisting of diabetic nephropathy, proteinuria, impaired glomerular clearance, retinopathy, neuropathy, atherosclerosis, diabetes-associated hyperlipidemia, oxidative modification of proteins, arthritis, connective tissue diseases, amyloidosis, urinary stone disease, obesity-related complications proliferation or smooth muscle cells in the aorta, coronary artery occlusion, and hypertension; and dialysis-related disorders including dialysis-related cardiac morbidity and mortality, dialysis-related amyloidosis, dialysis-related increases in permeability of the peritoneal membrane in a dialysis patient, renal failure progression in a dialysis patient, and inhibiting ultrafiltration failure and peritoneal membrane destruction in a dialysis patient, wherein the method comprises administering an effective amount of a pharmaceutical composition according to claim 26 to a subject in need of such treatment.
